(19) United States
(12) Patent Application Publication
(10) Pub. No.: US 2024/0062897 A1
(43) Pub. Date: Feb. 22, 2024

(54) ARTIFICIAL INTELLIGENCE METHOD FOR EVALUATION OF MEDICAL CONDITIONS AND SEVERITIES

(71) Applicant: Montera d/b/a Forta, San Francisco, CA (US)

(72) Inventors: Anurag GARIKIPATI, San Francisco, CA (US); Rahul THAPA, San Francisco, CA (US); Qingqing MAO, San Francisco, CA (US); Madalina CIOBANU, San Francisco, CA (US)

(21) Appl. No.: 17/890,971

(22) Filed: Aug. 18, 2022

(51) Int. Cl. G16H 50/20

(52) U.S. Cl. CPC G16H 50/20

(57) ABSTRACT

A method implemented via a computing device. The method may include receiving, by the computing device, data associated with a subject. The data associated with the subject may include two or more of demographic data, comorbidity data, observational assessment and interview data, and medication data. The method may also include evaluating, by the computing device, the data associated with the subject via an autism spectrum disorder (ASD) model. The ASD model may evaluate the data associated with the subject to determine the presence or absence of an ASD and, based upon a determination of the presence of an ASD, classify the ASD. The evaluation of the data associated with the subject by the ASD model may yield an evaluation result. The evaluation result may indicate the presence or absence of the ASD.

### TECHNICAL FIELD

The present disclosure relates to methods and systems pertaining to the use of artificial intelligence for the evaluation of medical conditions and severities including autism spectrum disorder (ASD).

### BACKGROUND

ASD is a complex neurodevelopmental disorder which expresses heterogeneously in afflicted individuals, although a few essential features are commonly present: social communication impairment as well as restricted interests and repetitive behaviors. It is estimated that currently about 1 in 100 children worldwide are diagnosed with ASD, while the Centers for Disease Control and Prevention (CDC) estimates based on 2018 data that about 1 in 44 8-year-old children have been identified with ASD in the United States. ASD occurs across all geographic regions and socio-economic groups. Generally, the discrepancies in officially diagnosed ASD from one demographic to another can largely be attributed to the difficulty of the diagnostic process, societal stigma, or lack of awareness, rather than an actual difference in disorder prevalence between demographics.

Conventionally, diagnosis of ASD is a challenging and elaborate process involving multiple phases and determination (e.g., differential diagnosis) by a clinician of ASD versus other developmental disorders. An accurate ASD diagnosis is difficult because ASD is generally enmeshed with other comorbidities, which can be of a neurodevelopmental or other medical nature. Further, ASD screening is not standardized during early childhood visits to a pediatrician, and the range of presentations make early diagnosis more challenging. Generally, an ASD diagnosis relatively earlier in life is associated with a relatively higher socio-economic status of the family, and African American and Hispanic children, for example, tend to be diagnosed at a relatively later age than their white counterparts. Generally, living in rural and other underserved communities often leads to a later ASD diagnosis.

Also, an early, accurate ASD diagnosis may be associated with better prognosis, for example, a better quality of life, ranging from significant gains in cognition, language, and adaptive behavior to more functional outcomes in later life. Furthermore, the ASD language deficit is intertwined on a fundamental level with the ability to diagnose other comorbidities. Individuals with ASD that either do not have an official diagnosis or are not benefitting from early intervention have a relatively higher degree of difficulty conveying their symptoms (owing to the language deficit), while tending to exhibit disruptive behaviors. This, in turn, may mask other neurodevelopmental and/or medical conditions which may remain undiagnosed, thus causing both short-term as well as long-term problems for the undiagnosed individual.

Given the complex and challenging nature of the diagnostic process for ASD, there is an ongoing need to develop methods for identifying and diagnosing individuals with ASD and, at the same time, for providing easier, more readily-available access to such methods.

### BRIEF SUMMARY

Disclosed herein is a method implemented via a computing device. The method may comprise receiving, by the computing device, data associated with a subject, the data associated with the subject comprising two or more of demographic data, comorbidity data, observational assessment and interview data, and medication data. The method may also comprise evaluating, by the computing device, the data associated with the subject via an autism spectrum disorder (ASD) model, wherein the ASD model is configured to evaluate the data associated with the subject to determine the presence or absence of an ASD and, based upon a determination of the presence of an ASD, classify the ASD, wherein evaluation of the data associated with the subject by the ASD model yields an evaluation result, wherein the evaluation result indicates the presence or absence of the ASD.

Additionally or alternatively, also disclosed herein is a computing system for evaluating a subject with respect to ASD. The system may comprise a computing device, the computing device comprising a processor and a non-transitory computer-readable medium. The non-transitory computer-readable medium includes instructions configured to cause the processor to implement an ASD model. The ASD model, when implemented via the processor, may cause the computing device to receive data associated with a subject, the data associated with the subject comprising two or more of demographic data, comorbidity data, observational assessment and interview data, and medication data. The ASD model, when implemented via the processor, may also cause the computing device to evaluate the data associated with the subject via an ASD model. The ASD model may be configured to evaluate the data associated with the subject to determine the presence or absence of an ASD and, based upon a determination of the presence of an ASD, classify the ASD. The evaluation of the data associated with the subject by the ASD model may yield an evaluation result. The evaluation result may indicate the presence or absence of the ASD.

Additionally or alternatively, also disclosed herein is a method implemented via a computing device. The method may comprise receiving, by the computing device, training data associated with a plurality of subjects, wherein at least a portion of the subjects are persons characterized as having an ASD, and wherein the training data associated with each of the subjects comprises two or more of demographic data, comorbidity data, observational assessment and interview data, and medication data. The method may also comprise processing the training data associated with the plurality of subjects to yield an ASD model, wherein the ASD model is configured to evaluate data associated with a subject to determine the presence or absence of an ASD and, based upon a determination of the presence of an ASD, classify the ASD, wherein evaluation of the data associated with the subject by the ASD model yields an evaluation result, wherein the evaluation result indicates the presence or absence of the ASD.

Additionally or alternatively, also disclosed herein is a method implemented via a computing device. The method may comprise receiving, by the computing device, data associated with a subject, the data associated with the subject comprising two or more of demographic data, comorbidity data, observational assessment and interview data, and medication data. The method may also comprise evaluating, by the computing device, the data associated with the subject via an ASD model, wherein the ASD model is configured to evaluate the data associated with the subject to yield an evaluation result, and wherein the evaluation result indicates a finding of non-ASD, a finding of autistic disorder, a finding of Asperger syndrome, or a finding of pervasive developmental disorder-not otherwise specified (PDD-NOS) for the subject.

### BRIEF DESCRIPTION OF THE DRAWINGS

For a detailed description of the preferred aspects of the disclosed processes and systems, reference will now be made to the accompanying drawings in which:

FIG. 1 displays a schematic diagram of an embodiment of the implementation of a model as disclosed herein;

FIG. 2A displays a schematic diagram of another embodiment of the implementation of a model as disclosed herein;

FIG. 2B displays a schematic diagram of yet another embodiment of the implementation of a model as disclosed herein;

FIG. 3 is a schematic representation of a computing system by way of which a machine-learning model may be employed;

FIG. 4 is a schematic representation of a machine-learning model;

FIG. 5 is a schematic diagram of an embodiment of methods related to a model as disclosed herein;

FIG. 6A is a diagram of certain results related to an embodiment of a model of the type disclosed herein;

FIG. 6B is a diagram of certain results related to another embodiment of a model of the type disclosed herein;

FIG. 7A is a diagram of certain results related to an embodiment of a model of the type disclosed herein;

FIG. 7B is a diagram of certain results related to another embodiment of a model of the type disclosed herein;

FIG. 8A is a diagram of certain results related to an embodiment of a model of the type disclosed herein;

FIG. 8B is a diagram of certain results related to another embodiment of a model of the type disclosed herein;

FIG. 9A is a diagram of certain results related to an embodiment of a model of the type disclosed herein;

FIG. 9B is a diagram of certain results related to another embodiment of a model of the type disclosed herein;

FIGS. 10A and 10B are diagrams of certain results related to an embodiment of a model of the type disclosed herein;

FIGS. 10C and 10D are diagrams of certain results related to an embodiment of a model of the type disclosed herein;

FIG. 11A is a diagram of certain results related to an embodiment of a model of the type disclosed herein; and

FIG. 11B is a diagram of certain results related to another embodiment of a model of the type disclosed herein.

### DETAILED DESCRIPTION

In various embodiments disclosed herein are methods, systems, and devices related to the identification and diagnosis of medical conditions and severities including a disorder on the autism spectrum or Autism Spectrum Disorder (ASD). For purposes of the disclosure herein, the terms “disorder on the autism spectrum” and “ASD” may be used interchangeably to refer to a disorder encompassing autistic disorder, Asperger's disorder, pervasive developmental disorder-not otherwise specified (PDD-NOS), or ASD, where such disorders meet the diagnostic criteria of an accepted or recognized standard fir diagnosis of the relevant disorder, for example, the Diagnostic and Statistical Manual of Mental Disorders, 4^{th} Edition (DSM-IV), the Diagnostic and Statistical Manual of Mental Disorders, Fifth Edition (DSM-5), both the DSM-IV and DSM-5, or a later iteration thereof. More particularly, the methods, systems, and devices disclosed herein may be effective or function to predict the presence or absence of an ASD with respect to a subject. Additionally, in some embodiments, the disclosed methods, systems, and devices may be effective to classify the ASD (e.g., into ASD subtypes, such as autistic disorder, Asperger's disorder, PDD-NOS, or some additional subclassification and/or to classify ASD vs. non-ASD). Further, and for purposes of the disclosure herein, the terms “non-ASD” and “non-spectrum” may be used interchangeably to refer to a lack of ASD.

In some embodiments, the disclosed methods, systems, and devices may implement a model effective to make one or more predictions about ASD with respect to the subject. Generally, the terms “subject” and “patient” may be used interchangeably to refer to a human involved in one or more aspects of the disclosed subject matter.

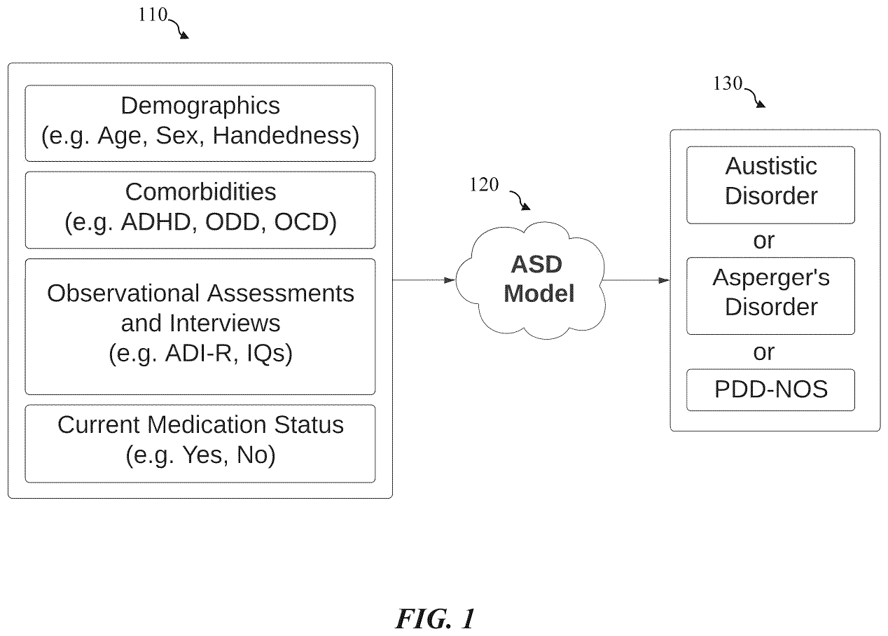

Referring to FIG. 1, an embodiment of the implementation of a model illustrated. For example, in the embodiment of FIG. 1, data associated with the subject, as will be disclosed herein, is utilized as inputs 110 by an ASD model 120. In the embodiment of FIG. 1, the subject may be characterized as having been previously identified as having an ASD. As will be discussed herein, the ASD model 120 is configured to evaluate the data associated with the subject to classify the ASD of the subject. For example, evaluation of the data associated with the subject by the ASD model 120 may yield an evaluation result 130 that indicates a classification of ASD as autistic disorder, Asperger syndrome, or PDD-NOS. For purposes of the disclosure herein, the terms “autistic disorder” and “autism” may be used interchangeably to refer to a disorder that meets the diagnostic criteria of the accepted or recognized standard for diagnosis of the relevant disorder, for example, the DSM-IV, the DSM-5, both the DSM-IV and DSM-5, or a later iteration thereof. Further, and for purposes of the disclosure herein, the terms “Asperger's disorder” and “Asperger syndrome” may be used interchangeably to refer to a disorder that meets the diagnostic criteria of the accepted or recognized standard for diagnosis of the relevant disorder, for example, the DSM-IV, the DSM-5, both the DSM-IV and DSM-5, or a later iteration thereof.

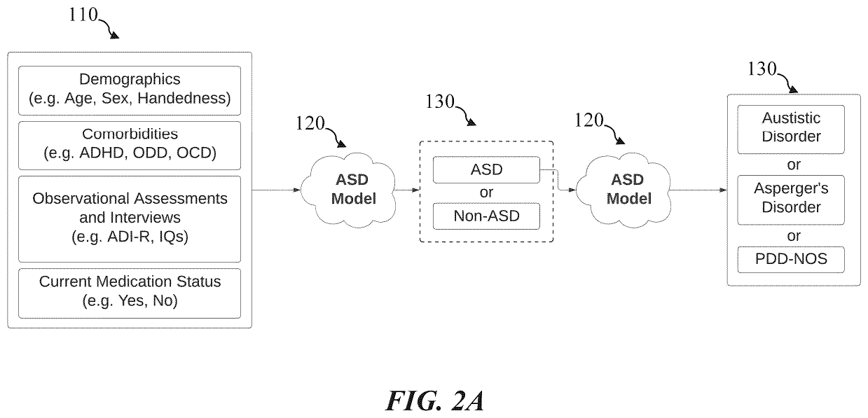

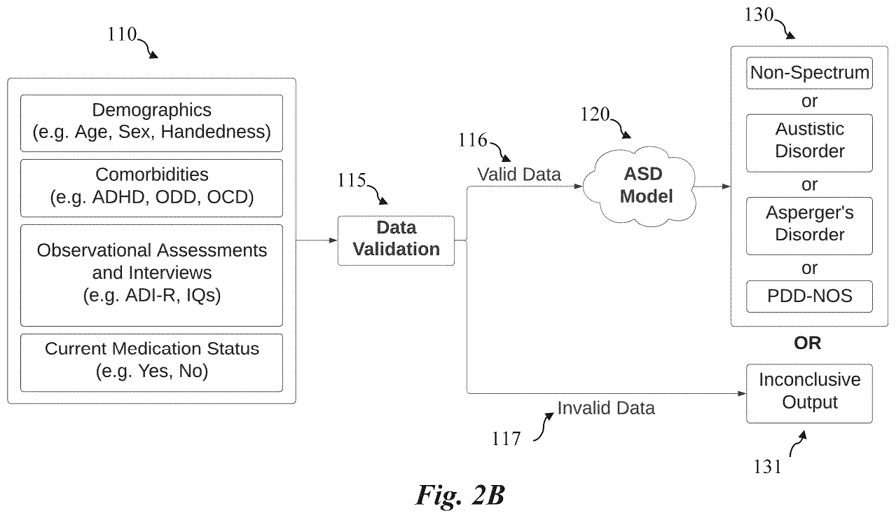

Additionally, referring to FIG. 2A, another embodiment of the implementation of the ASD model 120 is illustrated. In some embodiments of FIG. 2A, the subject may be characterized as not having been previously identified as having an ASD. In other embodiments of FIG. 2A, the subject may be characterized as having been previously identified as having an ASD, but not having an ASD subtype (e.g., autistic disorder, Asperger's disorder, PDD-NOS) associated therewith. In an embodiment of FIG. 2A, the ASD model 120 may be configured to evaluate the data associated with the subject to determine the presence or absence of an ASD and, based upon a determination of the presence of an ASD, classify the ASD. For example, evaluation of the data associated with the subject by the ASD model 120 may yield an evaluation result 130 that indicates both the presence or absence of the ASD and, if the evaluation result 130 indicates the presence of the ASD, the evaluation result 130 may further indicate a classification of the ASD, more particularly, as autistic disorder, Asperger syndrome, or PDD-NOS. In some aspects, the implementation of the ASD model 120 can comprise classifying a subject having an ASD diagnosis as having autistic disorder, Asperger syndrome, or PDD-NOS.

Additionally, referring to FIG. 2B, another embodiment of the implementation of the ASD model 120 is illustrated. In some embodiments of FIG. 2B, the subject may be characterized as not having been previously identified as having an ASD. In other embodiments of FIG. 2B, the subject may be characterized as having been previously identified as having an ASD, but not having been previously identified as having a particular ASD subtype (e.g., autistic disorder, Asperger's disorder, PDD-NOS) associated therewith. In an embodiment of FIG. 2B, the ASD model 120 may be configured to evaluate previously validated data (e.g., for example via a data validation step 115) associated with the subject to determine whether the subject is non-spectrum, has autistic disorder, has Asperger's disorder, or has PDD-NOS. For example, validation 115 of the data associated with the subject may separate valid data 116 from any other data such that only valid data 116 is then input into the ASD model 120. The ASD model 120 may be configured to evaluate the valid data 116 associated with the subject to determine whether the subject is classified as non-spectrum, has autistic disorder, has Asperger's disorder, or has PDD-NOS. Additionally or alternatively, as an example, validation 115 of the data associated with the subject may also separate invalid data 117 from any other data such that the invalid data 117 is not input into the ASD model 120, which could have the effect of leading to an inconclusive output 131. For purposes of the disclosure herein, the term “valid data” refers to data that can be evaluated by the ASD model 120 to determine (i) whether a subject has ASD or not; (ii) whether a subject should be classified as having autistic disorder, Asperger's disorder, or PDD-NOS; or (iii) combinations thereof. Further, and for purposes of the disclosure herein, the term “invalid data” refers to data that, if processed by the ASD model 120, may lead to an inconclusive, incorrect, or illogical result. For example, invalid data containing significant outliers may include data which is greater than about 1 standard deviation away from the mean of the entire dataset, alternatively greater than about 1.5 standard deviations away from the mean of the entire dataset, alternatively greater than about 2 standard deviations away from the mean of the entire dataset, or alternatively greater than about 1.5 times the interquartile range of the entire dataset. The invalid data may be, for example, (i) incomplete or insufficient for running the ASD model, (ii) data having significant outliers (e.g., values outside expected ranges; an IQ of 180 could be an outlier); (iii) or combinations thereof. For example, an inconclusive output 131 may further indicate to a healthcare professional or other user that the patient data may have been input incorrectly into the ASD model 120, and thus the inputs should be double checked and corrected; and/or the patient data may fall on outlier values for certain ranges, and thus the patient should undergo further assessment in an attempt to clarify the outlier values. In some cases, the assessments that yielded outlier values may be repeated for validation.

Additionally or alternatively, in some embodiments, the ASD model 120 may be configured to validate data associated with the subject, for example, such that only valid data 116 is considered by the ASD model 120 and/or such that invalid data 117 is disregarded by the ASD model 120.

In various embodiments, the data associated with the subject that is used as the input 110 to the ASD model 120 may comprise demographic data, comorbidity data, observational assessment and interview data, medication data, or combinations thereof. For example, in some embodiments, the data associated with the subject that is used as the input 110 to the ASD model 120 may comprise two or more of the demographic data, comorbidity data, observational assessment and interview data, medication data; additionally or alternatively, three or more of the demographic data, comorbidity data, observational assessment and interview data, medication data; additionally or alternatively, four or more of the demographic data, comorbidity data, observational assessment and interview data, medication data; additionally or alternatively, each of the demographic data, comorbidity data, observational assessment and interview data, medication data. Various examples of the various types and/or categories of the data associated with the subject are illustrated in Table 1:

TABLE 1

| Observational Assessment | | |
|---|---|---|
| and Interview Data | | Relevant Comorbidities Status |
| Autism Diagnostic Interview, Revised (ADR-I) | | Attention Deficit Hyperactivity |
| Social Total (e.g., social | | Disorder (ADHD) (yes or no) |
| communication total) | | Phobias (yes or no) |
| Verbal Total (e.g., verbal | | Oppositional Defiant Disorder (ODD) |
| communication total) | | (yes or no) |
| Restricted, Repetitive Behaviors Total | | Obsessive-Compulsive Disorder |
| Onset Total (the term “onset” refers to | | (OCD) (yes or no) |
| Abnormality of Development Evident | | Anxiety/Generalized Anxiety Disorder |
| at or Before 36 Months) | | (GAD) (yes or no) |
| Autism Diagnostic Observation Schedule, 1^{st} | | |
| and/or 2^{nd} Edition (ADOS and/or ADOS-2) | | |
| Social Responsiveness Scale (SRS) | | |
| Social Communication Questionnaire (SCQ) | | |
| Autism Screening Questionnaire (ASQ) | | |
| Vineland Adaptive Behavior Scale (VABS) | | |
| Behavior Rating Inventory of Executive | | |
| Function (BRIEF) | | |
| | Current Medication Status | Age |
| | Yes | Intelligence Quotient (IQ) |
| | No | Full-scale IQ (FIQ) |
| | If yes, medications and/or class of | Verbal IQ (VIQ) |
| | medication | Performance IQ (PIQ) |
| | | Sex |
| | | Male |
| | | Female |
| | | Handedness |
| | | Right |
| | | Left |
| | | Ambidextrous |

For example, the demographic data may include age data, intelligence quotient (IQ) data, sex data, handedness data, genetic data (e.g., presence or absence of single nucleotide polymorphisms (SNPs) and/or polygenetic risk scores), brain-imaging data, the like, or combinations thereof. Also for example, the comorbidity data may include an indication of the presence or absence of attention deficit hyperactivity disorder (ADHD), a phobia, obsessive-compulsive disorder (OCD), oppositional defiant disorder (ODD), anxiety, generalized anxiety disorder (GAD), intellectual disability (ID), bipolar disorder, communication disorders, speech or language disorders, motor disorders, neurogenetic disorders (e.g., Down syndrome, Rett syndrome), specific learning disorders (e.g., dyslexia), traumatic brain injury (TBI), fetal alcohol spectrum disorders (FASD), the like, or combinations thereof. Also for example, the observational assessment and interview data may include Autism Diagnostic Instrument-Revised (ADI-R) data, Autism Diagnostic Observation Schedule (ADOS) 1^{st} and/or 2^{nd} Edition (ADOS and/or ADOS-2) data, Social Responsiveness Scale (SRS) data, Social Communication Questionnaire (SCQ) data, Autism Screening Questionnaire (ASQ) data, Vineland Adaptive Behavior Scale (VABS) data, Behavior Rating Inventory of Executive Function (BRIEF) data, or combinations thereof. Also for example, the medication data may include an indication of any medications used by the subject.

In some embodiments, the data associated with the subject may be configured for input into a computing system, for example, such that the data associated with the subject may be evaluated via the ASD model. The data associated with the subject, also referred to as data features, may be represented and/or formatted in any suitable way. For example, the data associated with the subject comprises structured data, that is, data having a standardized format. Data feature processing can be performed prior to inputting the data into the ASD model (e.g., ASD model 120). For example, some text data may be converted into binary (true vs. false, yes vs. no) data to display the presence or the absence of a certain feature for a particular subject. In some aspects, two or more input features may be combined prior to input into the ASD model. In other aspects, two or more input features may be combined subsequent to input into the ASD model. In yet other aspects, some input features may be combined prior to input into the ASD model; and other input features may be combined subsequent to input into the ASD model.

As an example of the ways in which various of the data associated with the subject may be configured for input, age may be represented in the number of years. IQ may also be represented numerically. Additionally, IQ may be subdivided into two or more categories: verbal, performance, full-scale, or combinations thereof; with Verbal IQ being a measure of the subject's overall verbal intellectual abilities including acquired knowledge, verbal reasoning, and attention to verbal materials, performance IQ being a measure of the subject's overall visuospatial intellectual abilities, and full-scale IQ being a measure of the subject's overall level of general cognitive and intellectual functioning. The IQ data may be obtained via various assessment tools such as the Wechsler Abbreviated Scales of Intelligence (WASI), the Wechsler Intelligence Scale for Children (WISC), the Stanford Binet Intelligence Scales, and the like. The observational assessment and/or interview data may also be represented numerically. For example, ADI-R may entail the aggregation of four different scores: social total, verbal total, restricted repetitive behaviors total, and onset total. For instance, the social total may be the reciprocal of a social interaction subscore; the verbal total may be a subscore pertaining to abnormalities in communication; restricted, repetitive behaviors may be a subscore pertaining to restricted, repetitive, and stereotyped patterns of behavior; and onset total may be a subscore pertaining to abnormalities of development evident at or before 36 months. Handedness may be represented numerically by conversion into 3 binary features representative of whether the candidate is left-handed, right-handed, or ambidextrous. Similarly, the sex feature may be represented numerically by binarization into male and female. Current medication status may be a Boolean feature with various “True” or “False” inputs based on the medication status of the candidate. Finally, relevant comorbidities may be represented as multiple, individual Boolean feature sets, or extracted from the free text as represented in their medical history, in the present example, such as ADHD, ODD, OCD, phobias, and GAD. In some embodiments, one or more features may include null values, for example, the IQ data and/or ADI-R data may contain null values, which may be handled implicitly by the ASD model.

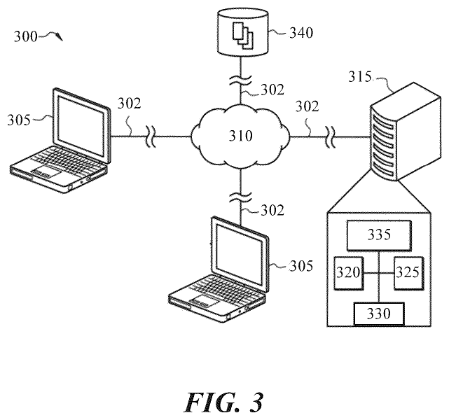

In some embodiments, the ASD model 120 may be characterized as a machine-learning model. An example of a machine-learning model, for example, the ASD model as disclosed herein is illustrated in the context of FIG. 3. For example, FIG. 3 illustrates an embodiment of a computing system 300 that includes a number of clients 305, a server system 315, and a data repository 340 communicably coupled through a network 310 by one or more communication links 302 (e.g., wireless, wired, or a combination thereof). The computing system 300, generally, can execute applications and analyze data received from sensors, such as may be acquired in the performance of the methods disclosed herein. For instance, the computing system 300 may execute a machine-learning model 335 as disclosed herein.

In general, the server system 315 can be any server that stores one or more hosted applications, such as, for example, the machine-learning model 335. In some instances, the machine-learning model 335 may be executed via requests and responses sent to users or clients within and communicably coupled to the illustrated computing system 300. In some instances, the server system 315 may store a plurality of various hosted applications, while in other instances, the server system 315 may be a dedicated server meant to store and execute only a single hosted application, such as the machine-learning model 335.

In some instances, the server system 315 may comprise a web server, where the hosted applications represent one or more web-based applications accessed and executed via network 310 by the clients 305 of the system to perform the programmed tasks or operations of the hosted application. At a high level, the server system 315 can comprise an electronic computing device operable to receive, transmit, process, store, or manage data and information associated with the computing system 300. The server system 315 illustrated in FIG. 3 can be responsible for receiving application requests from one or more client applications associated with the clients 305 of the computing system 300 and responding to the received requests by processing the requests in the associated hosted application and sending the appropriate response from the hosted application back to the requesting client application.

In addition to requests from the clients 305, requests associated with the hosted applications may also be sent from internal users, external or third-party customers, other automated applications, as well as any other appropriate entities, individuals, systems, or computers. As used in the present disclosure and as described in more detail herein, the term “computer” is intended to encompass any suitable processing device, such as an electronic computing device. For example, although FIG. 3 illustrates a single server system 315, a computing system 300 can be implemented using two or more server systems 315, as well as computers other than servers, including a server pool. The server system 315 may be any computer or processing device such as, for example, a blade server, general-purpose personal computer (PC), Macintosh, workstation, UNIX-based workstation, or any other suitable device. In other words, the present disclosure contemplates computers other than general-purpose computers, as well as computers without conventional operating systems. Further, the illustrated server system 315 may be adapted to execute any operating system, including Linux, UNIX, Windows, MacOS, or any other suitable operating system. In some embodiments, the server system 315 comprises a cloud-based server, an edge server, or a combination thereof. For example, the electronic computing device may comprise an edge computing device, a cloud computing device, or both.

In the illustrated embodiment, and as shown in FIG. 3, the server system 315 includes a processor 320, an interface 330, a memory 325, and the machine-learning model 335. The interface 330 is used by the server system 315 for communicating with other systems in a client-server or other distributed environment (including within computing system 300) connected to the network 310 (e.g., clients 305, as well as other systems communicably coupled to the network 310). Generally, the interface 330 comprises logic encoded in software and/or hardware in a suitable combination and operable to communicate with the network 310. More specifically, the interface 330 may comprise software supporting one or more communication protocols associated with communications such that the network 310 or interface's hardware is operable to communicate physical signals within and outside of the illustrated computing system 300.

Although illustrated as a single processor 320 in FIG. 3, two or more processors may be used according to particular needs, desires, or particular embodiments of computing system 300. Each processor 320 may be a central processing unit (CPU), a blade, an application-specific integrated circuit (ASIC), a field-programmable gate array (FPGA), or another suitable component. Generally, the processor 320 executes instructions and manipulates data to perform the operations of server system 315 and, specifically, the machine-learning model 335. Specifically, the server's processor 320 executes the functionality required to receive and respond to requests from the clients 305 and their respective client applications, as well as the functionality required to perform the other operations of the machine-learning model 335.

Regardless of the particular implementation, “software” may include computer-readable instructions, firmware, wired or programmed hardware, or any combination thereof on a tangible medium operable when executed to perform at least the processes and operations described herein. Each software component may be fully or partially written or described in any appropriate computer language including C, C++, C #, Java, Visual Basic, assembler, Perl, any suitable version of 4GL, Python, as well as others. It will be understood that while portions of the software implemented in the context of the embodiments disclosed herein may be shown as individual modules that implement the various features and functionality through various objects, methods, or other processes, the software may instead include a number of sub-modules, third-party services, components, libraries, and such, as appropriate. Conversely, the features and functionality of various components can be combined into single components as appropriate. In the illustrated computing system 300, processor 320 executes one or more hosted applications on the server system 315.

At a high level, the machine-learning model 335 is any application, program, module, process, or other software that may execute, change, delete, generate, or otherwise manage information according to the present disclosure, particularly in response to and in connection with one or more requests received from the illustrated clients 305 and their associated client applications. In certain cases, only one machine-learning model 335 may be located at a particular server system 315. In others, a plurality of related and/or unrelated modeling systems may be stored at a server system 315, or located across a plurality of other server systems 315, as well. In certain cases, computing system 300 may implement a composite hosted application. For example, portions of the composite application may be implemented as Enterprise Java Beans (EJBs) or design-time components may have the ability to generate run-time implementations into different platforms, such as J2EE (Java 2 Platform, Enterprise Edition), ABAP (Advanced Business Application Programming) objects, or Microsoft's .NET, among others. Additionally, the hosted applications may represent web-based applications accessed and executed by clients 305 or client applications via the network 310 (e.g., through the Internet).

Further, while illustrated as internal to server system 315, one or more processes associated with machine-learning model 335 may be stored, referenced, or executed remotely. For example, a portion of the machine-learning model 335 may be a web service associated with the application that is remotely called, while another portion of the machine-learning model 335 may be an interface object or agent bundled for processing at a client 305 located remotely. Moreover, any or all of the machine-learning model 335 may be a child or sub-module of another software module or enterprise application (not illustrated) without departing from the scope of this disclosure. Still further, portions of the machine-learning model 335 may be executed by a user working directly at server system 315, as well as remotely at clients 305.

The server system 315 also includes memory 325. Memory 325 may include any memory or database module and may take the form of volatile or non-volatile memory. The illustrated computing system 300 of FIG. 3 also includes one or more clients 305. Each client 305 may be any computing device operable to connect to or communicate with at least the server system 315 and/or via the network 310 using a wired or wireless connection.

The illustrated data repository 340 may be any database or data store operable to store data, such as data of the type disclosed herein as associated with one or more subjects. Generally, the data may comprise inputs to the machine-learning model 335, historical information, operational information such as features, and/or output data from the machine-learning model 335.

The functionality of one or more of the components disclosed with respect to FIG. 3, such as the server system 315 or the clients 305, can be carried out on a computer or other device comprising a processor (e.g., a desktop computer, a laptop computer, a tablet, a server, a smartphone, smartwatch, or some combination thereof). Generally, such a computer or other computing device may include a processor (which may be referred to as a central processor unit or CPU) that is in communication with memory devices including secondary storage, read-only memory (ROM), random access memory (RAM), input/output (I/O) devices, and network connectivity devices. The processor may be implemented as one or more CPU chips.

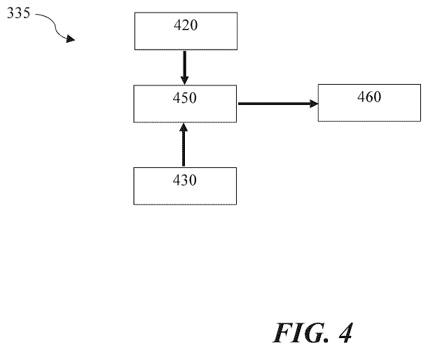

FIG. 4 depicts an example of the machine-learning model 335 of FIG. 3. In the embodiment of FIG. 4, the machine-learning model 335 comprises a machine-learning module 450 coupled to one or more data stores, for example, data within the data repository 340. For instance, in the embodiment of FIG. 4, the data within the data repository 340 of FIG. 3 may include data from a training data store 420 and/or other inputs 430, as will be disclosed herein.

The machine-learning module 450 can access data, such as data from the training data store 420, and receive inputs 430, and provide an output 460 based upon the inputs 430 and data retrieved from the training data store 420. Generally, the machine-learning module 450 utilizes data stored in the training data store 420, for example, data of the type disclosed herein as data associated with a subject, to enable the resulting trained model (for example, the ASD model disclosed herein) to evaluate data associated with a subject, for example, to predictively determine the presence or absence of an ASD and, based upon a determination of the presence of an ASD, to classify the ASD. For example, in some embodiments the ASD model may determine the presence of an ASD and a classification of the ASD. Alternatively, in some embodiments the ASD model may determine the absence of ASD, for example, a determination of non-ASD. For example, the trained model may, in some embodiments, be characterized as a prediction algorithm.

Generally, the machine-learning module 450 is a learning machine exhibiting “artificial intelligence” capabilities. For example, the machine-learning module 450 may utilize algorithms to learn via inductive inference based on observing data that represents incomplete information about statistical phenomenon and generalizes it to rules and to make predictions on missing attributes or future data. Further, the machine-learning module 450 may perform pattern recognition, in which the machine-learning module 450 “learns” to automatically recognize complex patterns, to distinguish between exemplars based upon varying patterns, and to make intelligent predictions. In some embodiments, the machine-learning module 450 can include or be accompanied by an optimization algorithm, like genetic algorithm (GA), ant colony optimization algorithm (ACO), simulated annealing (SA), etc. to increase the model accuracy and narrow down the data used to allow the machine-learning module 450 to operate efficiently, even when large amounts of historical training data are present, and/or when complex input parameters are present.

The machine-learning module 450 can comprise and/or implement any suitable machine-learning algorithm or methodology, examples of which may include, but are not limited to, artificial neural networks (ANNs), deep neural networks (DNNs), deep reinforcement learning, convolutional neural networks (CNNs), a deep learning model, a generative adversarial network (GAN) model, a computational neural network model, a recurrent neural network (RNN) model, a perceptron model, decision trees such as a classical tree machine-learning model, a decision tree type model, support vector machines, a regression type model, a classification model, a reinforcement learning model, Bayesian networks, optimization algorithms, and the like, and combinations thereof.

For example, in a particular embodiment, the machine-learning module 450 utilizes gradient-boosted tree machine learning, for example, implemented in Python. Generally, a gradient-boosted trees aggregate results from various decision trees to output prediction scores. A dataset being evaluated may be split into successively smaller groups within each decision tree, for example, such that each tree branch divides a subject into one of two groups according to their covariate value and a predetermined threshold. The end of the decision tree is a set of leaf nodes, each of which represents ASD patients (e.g., patients having or being suspected of having ASD). As the model is trained, successive trees are developed in order to improve the accuracy of the model. Successive iterations of trees utilize gradient descent of the prior trees in order to minimize the error of the new tree that is formed. In some embodiments, gradient-boosted tree machine learning implicitly handles any missing values, for example, various data associated with a subject that are not present. For instance, during the training phase, the model “learns” the optimal branch directions for missing values.

At a high level, the machine-learning module 450 may receive inputs 430 comprising constraints and parameters as to the training of the machine-learning model, to perform learning with respect to the training data. For example, in some embodiments, the machine-learning module 450 may “learn” or be trained by processing the training data, more particularly, the data from the training data store 420. As the machine-learning module 450 processes the training data, the machine-learning module 450 may form one or more probability-weighted associations between the various known inputs and the respective outcomes. As training progresses, the machine-learning module 450 may adjust weighted associations between various inputs, for example, according to a learning rule, in order to decrease the error between the inputs and their respective outputs. As such, the machine-learning module 450 may increasingly approach target output(s) until the error is acceptable.

In some embodiments, at least a portion of the data stored in the training data store 420 may be characterized as “training data” that is used to train the machine-learning model 335. As will be appreciated by the ordinarily-skilled artisan upon viewing the instant disclosure, although the Figures illustrate an aspect in which the training data are stored in a single “store” (e.g., at least a portion of the training data store 420), additionally or alternatively, in some embodiments the training data may be stored in multiple stores in one or more locations.

Additionally, in some embodiments, the training data (e.g., at least a portion of the data stored in the training data store 420) may be subdivided into two or more subgroups, for example, a training data subset, one or more evaluation and/or testing data subsets, or combinations thereof. The training data may include a plurality of batches of data, each batch representing a data for each of a plurality of scenarios. Each batch of data may include data associated with each of a plurality of training subjects, particularly, including known inputs (e.g., demographic data, comorbidity data, observational assessment and interview data, and medication data, as disclosed herein) associated with known outcome(s) (e.g., whether a training subject has an ASD and, if so, the classification of the ASD).

In various embodiments, the training data may include data associated with a plurality of subjects (e.g., training subjects), generally including data of the type disclosed herein as data associated with a subject, more particularly, demographic data, comorbidity data, observational assessment and interview data, and/or medication data. Additionally, the training data may also include an indication of whether a training subject has an ASD and, if so, the classification of the ASD. For example, the data employed as training data may be taken from a publicly available dataset that includes, for instance, subject demographic information, various diagnostic test scores for ASD, patient IQ information, medication and subject comorbidity information. The data used may be anonymized (e.g., de-identified), for example, to ensure compliance with various regulations concerning patient information. In addition, the dataset can also contain professional (e.g., “official”) diagnosis of ASD vs. non-ASD, as well as different sub-classes of ASD according to Diagnostic and Statistical Manual of Mental Disorders, 4^{th} Edition (DSM-IV) criteria, that is, a classification as to Autistic disorder, Asperger syndrome, or PDD-NOS.

In some embodiments, the inputs 430 can comprise one or more constraints or limitations that may affect the way in which the machine-learning module 450 is trained, an example of which includes various hyperparameters. In various embodiments, the inputs 430 can be provided as separate inputs, as a single input, or as a vector or matrix of input values. In some embodiments, the inputs 430 may be received, for example, from a user. Based on the inputs 430, the machine-learning module 450 may use the data stored in the training data store 420 to develop the machine-learning model 335, such as the ASD model 120 disclosed herein with respect to FIGS. 1 and 2.

As such, in some embodiments, based on processing the training data, for example, data from the training data store 420, the machine-learning module 450 may yield a trained machine-learning model 335 (e.g., the ASD model 120) that is configured to evaluate data associated with the subject to determine the presence or absence of an ASD and, based upon a determination of the presence of an ASD, classify the ASD.

In some embodiments, the ASD model may be configured to output a score, for example, between 0 and 1, indicative of the result. By default, a threshold of 0.5 is used to differentiate between positive and negative class, meaning that if the ASD model outputs a score greater than or equal to 0.5 for a subject, the subject belongs within the positive class, indicating that the individual is likely to have ASD and if the ASD model outputs a score less than 0.5, the subject belongs to the negative class, indicating that the individual is unlikely to have ASD. However, this threshold value does not have to be set to 0.5. A threshold value can be any suitable value between 0 and 1, for example a threshold value effective to yield a desired sensitivity. For example, the threshold value can be 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, or any other suitable fractional value between 0 and 1 that is effective to yield a desired sensitivity. In some embodiments, a threshold value can be a value effective to yield a desired sensitivity; for example a sensitivity from about 0.7 to about 1.0, alternatively from about 0.71 to about 0.99, alternatively from about 0.75 to about 0.95, alternatively from about 0.75 to about 0.99, alternatively from about 0.8 to about 0.95, alternatively from about 0.85 to about 0.95, alternatively equal to or greater than about 0.7, alternatively equal to or greater than about 0.8, or alternatively equal to or greater than about 0.9, for ASD vs. non-ASD classification.

In an embodiment, for example, where the machine-learning module 450 utilizes gradient-boosted tree machine learning, the trained machine-learning model 335 may be characterized as having a depth of at least 2 and not more than 10, additionally or alternatively, a depth of at least 2 and not more than 7, additionally or alternatively, a depth of not more than 6, additionally or alternatively, a depth of not more than 5, additionally or alternatively, a depth of not more than 4, additionally or alternatively, a depth of not more than 3. Additionally or alternatively, the gradient-boosted tree model comprises a plurality of decision trees, for example, at least 200 decision trees, additionally or alternatively, at least 250 decision trees, additionally or alternatively, at least 300 decision trees, additionally or alternatively, at least 350 decision trees, additionally or alternatively, at least 400 decision trees, additionally or alternatively, at least 450 decision trees, additionally or alternatively, at least 500 decision trees, additionally or alternatively, from about 200 to about 600 decision trees.

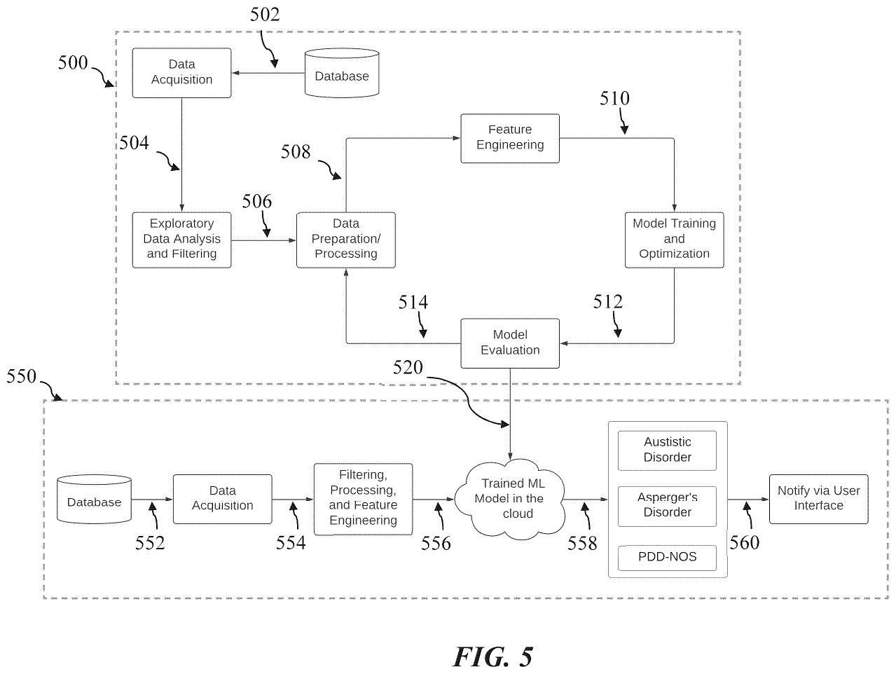

FIG. 5 illustrates methods related to the ASD model 120. Particularly, FIG. 5 illustrates both a method of training the ASD model 500 and a method of using the ASD model 550, for instance, in the identification, diagnosis, and/or classification of ASD.

Referring to FIG. 5, during the training phase 500, training data (e.g., a dataset) is acquired from the database (step 502). As disclosed herein, the training data may include patient health records including their demographics, relevant comorbidities, assessment and observational scores, medication information, and the like. The dataset may undergo exploratory data analysis, for example, which may be effective to evaluate the structure, distribution, and/or quality of the dataset (step 504). Additionally, the dataset may be processed, for example, filtered, to ensure that patient data that is severely out of distribution or has significant missingness which may skew the results is omitted. Additionally, for example, following this initial filtration, the training data may undergo further processing, including imputing or removing outliers and/or unidentified characters, and removing features that may be highly correlated with other features, have a high degree of missing values, or are not important (step 506). Additional features may also be extracted based upon examination of feature importance during preliminary model development, through advice/consultation on clinical judgment from experts in the field, or through some combination thereof. For example, textual data may be converted into binary features or categorical features may be broken into multiple binary features (step 508). The dataset may then be randomly divided into a training subset and a testing subset. For example, a hold-out test set may be formed including a random percentage of the individuals from the total dataset (e.g., from about 15% to about 25%, alternatively from about 16% to about 24%, alternatively from about 17% to about 23%, alternatively from about 18% to about 22%, alternatively from about 19% to about 21%, or alternatively about 20% of the individuals from the total dataset). The testing subset may be maintained completely independent of the training process such that it is solely used to evaluate the performance of the resultant model so as to determine the model's efficacy.

In some embodiments, prior to training the model, one or more hyperparameters may be optimized, for example, to determine the best hyperparameters for training utilizing a gradient-boosted tree learning model. Generally, hyperparameters are elements of the machine-learning model that dictate the training process and the specific way in which a machine-learning model learns. As an example, in gradient-boosted tree learning, the depth of each tree (meaning how many features are evaluated to classify a data input) is a hyperparameter of the model. Different tree depths would alter the way in which model training occurs so a hyperparameter optimization might evaluate depths of 2, 3, 4, 5, 6, 7, or more to identify which leads to optimal model performance. The hyperparameter optimization may also include a multiple-fold cross-validation, for example, in order to evaluate the hyperparameter's performance on unseen data. The cross-validation may include 2, 3, 4, 5, 6, 7, or more folds. Following hyperparameter optimization, the model may be trained, for example, using the optimized hyperparameters (step 510). In order to evaluate the model's performance, the hold-out test subset may be passed through the model and the results may be analyzed. In some embodiments, the hold-out data subset may be exclusively used to evaluate the performance results, for example, in order to prevent any data leakage from the training data on the model's performance (step 512). Based on the results that we get from this evaluation, the steps associated with data preparation and processing, feature engineering, model training, and evaluation may be repeated (step 514) iteratively until satisfactory performance is demonstrated (steps 508, 510, 512, and 514), for example, as demonstrated by a desired sensitivity and/or specificity.

When the trained model demonstrates satisfactory performance, the model may then be deployed, for example, into a cloud server, at which time the model is able to be used during prospective settings (step 520). During the prospective setting, data associated with a subject to be evaluated may be acquired, for example, from the database and then cleaned and processed as similarly done with respect to the training data (steps 552 and 554). The data associated with the subject being evaluated may then be input into the machine-learning model (for example, an ASD model served via a cloud device) to output a prediction, for example, a result including the ASD subclassification (steps 556 and 558). The prediction (e.g., the result) may be displayed to the end user, which usually is the person evaluating the subject (step 560), for example, a healthcare provider. The prediction may be presented to a user (e.g., a healthcare professional) via a user interface. For example, the prediction may be presented graphically, in written text, and/or as audio. In various embodiments, the user interface may include a graphical user interface (e.g., a screen and/or touch-screen), a speaker, or the like. For example, the user interface may be delivered via a user device (e.g., a desktop computer, a laptop computer, a tablet, a server, a smartphone, a smartwatch, or some combination thereof).

Additionally, in some embodiments, a method of using the ASD model may further include providing treatment to the subject receiving a diagnostic indication of either Autistic disorder, Asperger syndrome, or PDD-NOS under DSM-IV. In various embodiments, the treatment provided to the subject can comprise applied behavior analysis (ABA) therapy, speech therapy, physical therapy, and the like, or combinations thereof.

The ASD model as disclosed herein may be advantageously employed in the identification and diagnosis of medical conditions and severities including ASD. For instance, the ASD model demonstrates the unique potential to improve the ASD diagnostic process across all age groups, and further, for individuals that would have qualified for a diagnosis of ASD (e.g., Asperger syndrome, PDD-NOS) under DSM-IV, but would have failed to meet the narrower diagnostic criteria of Diagnostic and Statistical Manual of Mental Disorders, 5^{th} Edition (DSM-5). For instance, the ASD model bridges the diagnostic gap between DSM-IV and DSM-5. There is an unmet need for improved ASD diagnosis across all age groups, and further for individuals that would have qualified for a diagnosis of ASD (e.g., Asperger syndrome, PDD-NOS) under DSM-IV, but fail to meet the narrower diagnostic criteria of DSM-5.

Additionally, the disclosed ASD model allows for accurate identification of ASD and classification of sub-category of ASD in a subject, which conventionally involves a time-consuming and resource-intensive process, that can be achieved in a matter of minutes. Moreover, the disclosed ASD model empowers caregivers to detect a disorder much earlier than previously possible and plan an intervention with therapy as early as possible, which is highly desirable in the treatment of ASD disorders such as autism, Asperger syndrome, PDD-NOS, etc.

### EXAMPLES

The presently disclosed subject matter having been generally described, the following examples are given as particular aspects of the subject matter and to demonstrate the practice and advantages thereof. It is understood that the examples are given by way of illustration and are not intended to limit the specification or the claims in any manner. The investigations in the Examples herein have been conducted on two different datasets: a general population dataset encompassing both children, as well as adults, and a pediatric dataset encompassing only individuals under the age of 21.

### Example 1

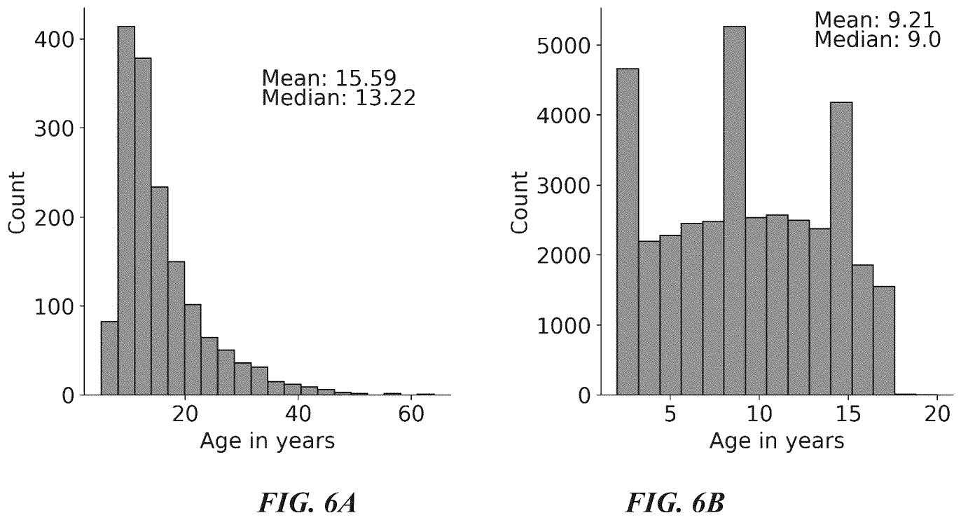

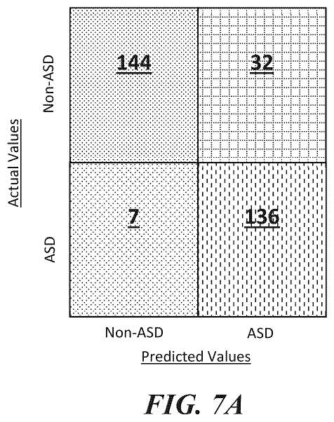

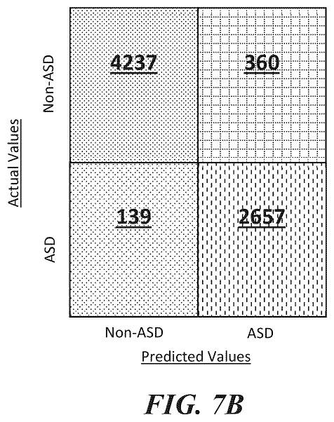

FIGS. 6A and 7A illustrate results of the operation of a machine-learning model of the type disclosed herein, particularly, operated to differentiate ASD and non-ASD in subjects. The data in FIGS. 6A and 7A are for the general population dataset. Particularly, FIG. 6A is a histogram plot showing the distribution of age in the entire population (both ASD and non-ASD). A histogram divides the variables into bins, counts the data points in each bin, and shows the bins on the x-axis and the counts on the y-axis. In this case, the bins represent the interval of age and the count is the number of flights falling into that interval. As shown, the histogram is right-skewed, meaning that the frequencies of the age are lower on the right side of the graph than the frequencies of the age on the left side of the graph. In FIG. 6A, there appears to be a normal distribution from the left side of the plot up until age 20 and the plot then has a long tail that extends to age 65, indicating that the majority of the subjects evaluated are under the age of 20 or not adults.

FIG. 7A illustrates a confusion matrix that summarizes the prediction results of the ASD model with respect to classification of ASD vs. non-ASD populations. The number of correct and incorrect predictions are presented with count values and broken down by each class. On the x-axis are the predicted values, meaning the predictions made by the ASD model, and on the y-axis are the actual values, or the ground truth. The confusion matrix provides insight into the errors being made by the classification model (i.e., the ASD model) as well as the types of errors that are being made. The top left and bottom right boxes represent the predictions where the ASD model predicted correctly, whereas the top right and bottom left boxes represent the examples where the ASD model did not predict correctly (or was “confused”). Below is the actual break down of the four categories:
Top left: True Negative
Bottom right: True Positive
Top right: False Positive
Bottom left: False Negative

For the same testing data and the same ASD model, multiple confusion matrices can be generated by operating the ASD model at different operating thresholds. By default, the operating threshold is 0.5. In the instant example, for this binary classification of the general population dataset problem, a threshold of 0.10 was chosen, or an operating sensitivity of approximately 0.95. An operating sensitivity refers to the sensitivity value at which the model is chosen to operate. For a specified sensitivity of interest, a threshold for the model can be selected which achieves said sensitivity. At this operating threshold, the ASD model performs very well, with only 39 misclassifications in 319 classifications.

Similarly to the data displayed in FIGS. 6A and 7A for the general population dataset, experiments have been performed on the pediatric dataset, and the results are displayed in FIGS. 6B and 7B, respectively. The histogram in FIG. 6B displays a similar age distribution to FIG. 6A for the pediatric age range (under 21 years old) indicating that ASD was diagnosed more frequently in the 7-15 years of age range. For the binary classification of the pediatric dataset problem, a threshold of 0.10 was chosen, or an operating sensitivity of approximately 0.95. At this operating threshold, the ASD model performs very well, with only 499 misclassifications in 7,393 classifications.

### Example 2

AUROC stands for Area Under Receiver Operating Curve (ROC), which is a performance metric that is used to evaluate classification models. AUROC is a performance metric of discrimination, that is, it provides information about the model's ability to discriminate between classes (ASD vs. non-ASD). AUROC of 0.50 is equivalent to random coin flip or no discrimination, whereas an AUROC of 0.70 means that the model will correctly assign a higher absolute risk to a randomly selected patient with an event than to a randomly selected patient without an event.

The ROC curve is made by plotting True Positive Rate (Sensitivity) and the complement of False Positive Rate (1—Specificity) at different threshold values. Threshold value is a value that is used to separate the positive class and negative class. For example, the ASD model disclosed herein outputs a score in between 0 and 1. Therefore, there are infinite threshold values that can be used to differentiate between two classes. For example, if the threshold value is 0.5, patients with model output values greater than and equal to 0.5 are classified as positive class (ASD) and less than 0.5 is classified as negative class (non-ASD). Based on the selected threshold value, other metrics such as true positive rate (TPR) and false positive rate (FPR) also change. Sensitivity or TPR is the proportion of subjects who are indicated as positive among all those who actually have the condition. Specificity or FPR is the proportion of subjects who test positive among all those who actually do not have the condition. Positive predictive value (PPV) is the probability that following a positive test result, a subject will truly have that condition. Negative predictive value (NPV) is the probability that following a negative test result, a subject will truly not have that specific disease. Plotting TPR and FPR at different thresholds yields the ROC curve. The area under this 2-Dimensional ROC curve is, simply, the AUROC.

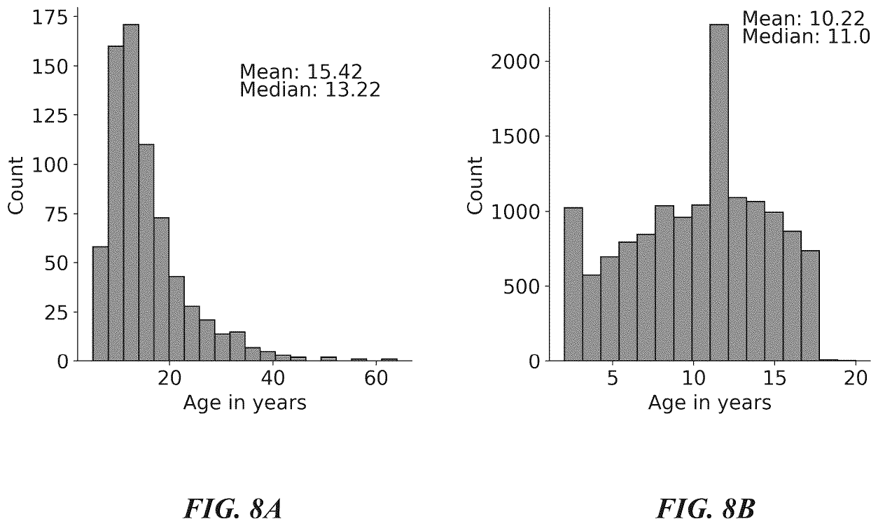

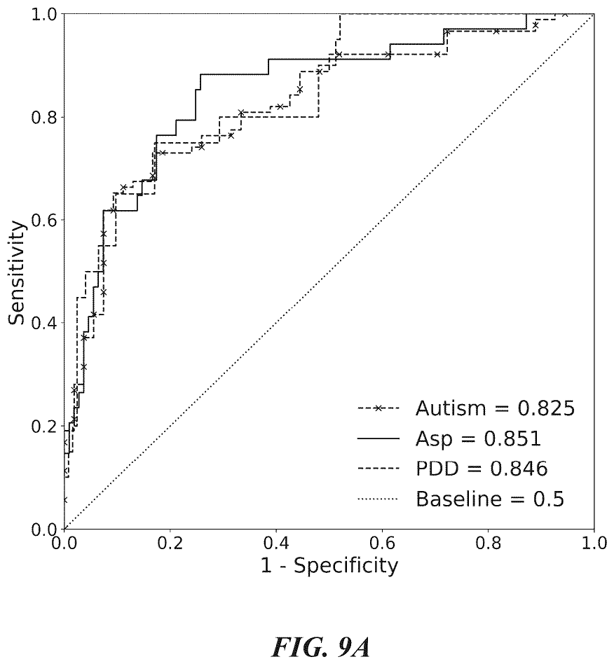

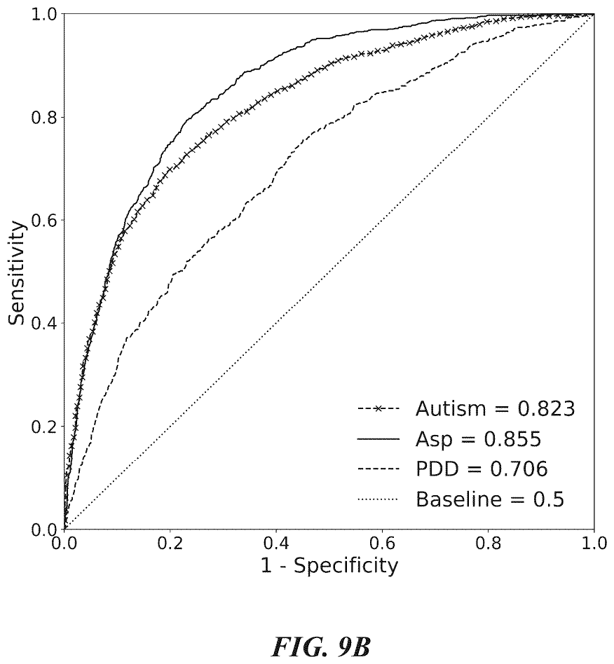

FIGS. 8A and 9A illustrate results of the operation of a machine-learning model of the type disclosed herein, particularly, operated to classify ASD subjects as having autism, Asperger syndrome, or PDD-NOS; wherein the subjects were part of the general population dataset. Particularly, FIG. 8A illustrates the histogram plot showing the distribution of age for just ASD population in the general population dataset (i.e., the subjects in the dataset that held a diagnosis of ASD). The distribution looks very similar to that of the total population, covering a wide range of age groups.

Table 2 is calculated based on the ROC curves (for the general population dataset) in FIG. 9A.

TABLE 2

| | Autism | Asperger Syndrome | PDD-NOS |
|---|---|---|---|
| AUROC | 0.825 | 0.851 | 0.846 |
| (95% Cl*) | (0.749-0.891) | (0.771-0.925) | (0.755-0.933) |
| Sensitivity | 0.764 | 0.765 | 0.750 |
| (95% CI) | (0.676-0.852) | (0.622-0.907) | (0.56-0.94) |
| Specificity | 0.741 | 0.826 | 0.829 |
| (95% CI) | (0.624-0.858) | (0.754-0.897) | (0.763-0.896) |

*CI = Confidence Interval

Referring to FIG. 9A, four (4) ROC curves are shown. The diagonal dotted line is a baseline with Area Under ROC (AUROC) of 0.5, which represents a model that is not able to differentiate between positive and negative classes at all, effectively equivalent to random coin-flip. The Autism curve in FIG. 9A is the ROC curve that represents the multi-class model's ability to differentiate an Autism subclass from other subclasses such as Asperger syndrome and PDD-NOS, for the general population dataset. Similarly, Asp and PDD-NOS in FIG. 9A are the ROC curves that represent the ASD model's ability to differentiate the Asperger syndrome and PDD-NOS subclasses respectively from the rest of the two subclasses, for the general population dataset.

Similarly to the data displayed in FIGS. 8A and 9A for the general population dataset, experiments have been performed on the pediatric dataset, and the results are displayed in FIGS. 8B and 9B, respectively. FIGS. 8B and 9B illustrate results of the operation of a machine-learning model of the type disclosed herein, particularly, operated to classify ASD subjects as having autism, Asperger syndrome, or PDD-NOS; the subjects were part of the pediatric dataset. Particularly, FIG. 8B illustrates the histogram plot showing the distribution of age for just ASD population in the pediatric dataset (i.e., the subjects in the dataset that held a diagnosis of ASD). The distribution looks very similar to that of the total pediatric population.

Table 3 is calculated based on the ROC curves (for the pediatric dataset) in FIG. 9B.

TABLE 3

| | Autism | Asperger Syndrome | PDD-NOS |
|---|---|---|---|
| AUROC | 0.823 | 0.855 | 0.706 |
| (95% Cl) | (0.808-0.838) | (0.838-0.87) | (0.682-0.728) |
| Sensitivity | 0.751 | 0.752 | 0.751 |
| (95% CI) | (0.727-0.775) | (0.724-0.78) | (0.717-0.785) |
| Specificity | 0.743 | 0.800 | 0.552 |
| (95% CI) | (0.721-0.764) | (0.782-0.818) | (0.531-0.573) |

Referring to FIG. 9B, four (4) ROC curves are shown, similarly to FIG. 9A. The Autism curve in FIG. 9B is the ROC curve that represents the multi-class model's ability to differentiate an Autism subclass from other subclasses such as Asperger syndrome and PDD-NOS, for the pediatric dataset. Similarly, Asp and PDD-NOS in FIG. 9B are the ROC curves that represent the ASD model's ability to differentiate the Asperger syndrome and PDD-NOS subclasses respectively from the rest of the two subclasses, for the pediatric dataset.

### Example 3

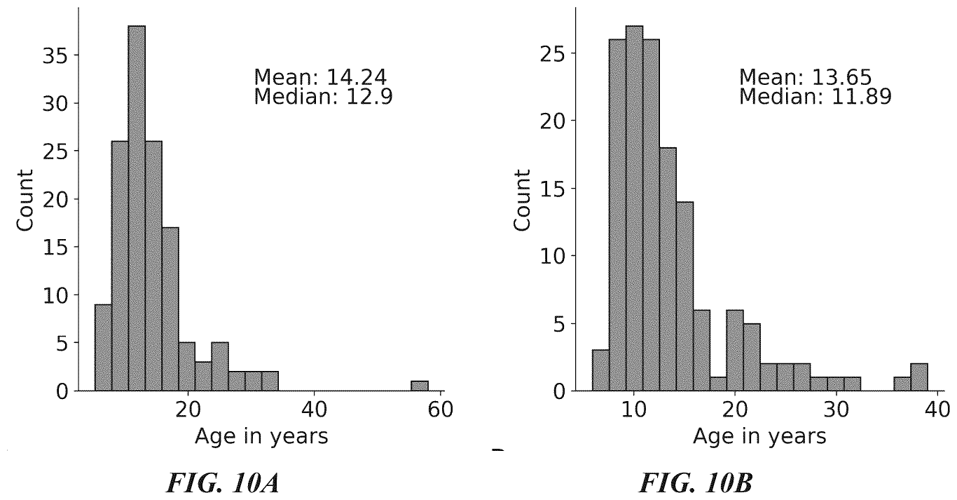

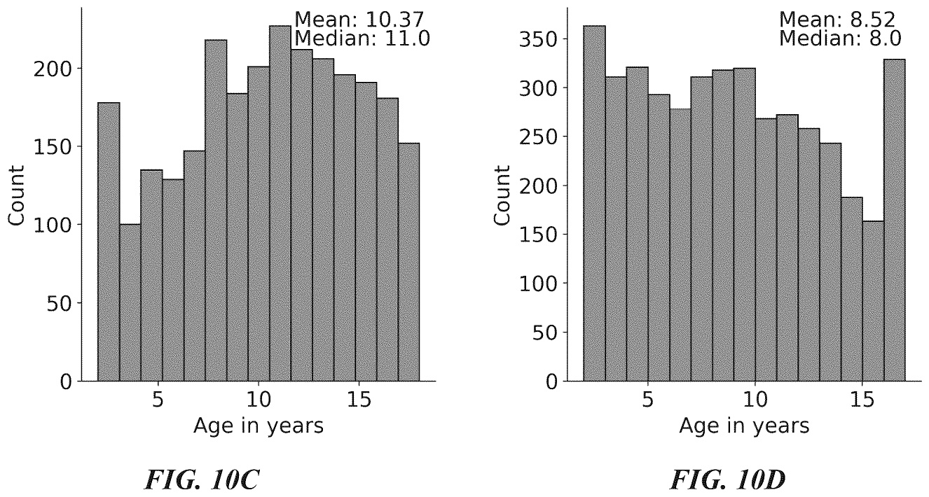

FIGS. 10A and 10B illustrate the results of a binary classification model that was developed to classify individuals with ASD vs. a control group of individuals without ASD, for the general population dataset. The performance of the model showcased high predictive capability to differentiate between the two groups with the performance metrics for that binary classification model (an ASD model) as displayed in Table 4 below.

TABLE 4

| Performance Metrics | Value |
|---|---|
| AUROC (95% CI) | 0.972 (0.954-0.988) |
| Sensitivity (95% Cl) | 0.951 (0.947-0.955) |
| Specificity (95% CI) | 0.818 (0.812-0.824) |
| PPV (95% CI) | 0.810 (0.804-0.816) |
| NPV (95% CI) | 0.954 (0.950-0.957) |

FIG. 10A illustrates a histogram plot showing the distribution of age for the population with a clinical diagnosis of ASD and, also, the predicted population by the classification model as having ASD (i.e., true positive values, that is, the population correctly predicted by the ASD model as having ASD); for the general population dataset. The oldest individual (58 years old) clinically diagnosed with ASD that was part of the testing group analyzed by the ASD model was also correctly identified by the classification model as having ASD (as shown in FIG. 10A). Further, FIG. 10B illustrates a histogram plot showing the distribution of age for the population without a clinical diagnosis of ASD and also predicted by the ASD model as not having ASD (i.e., true negative values, that is, the population correctly predicted by the ASD model as not having ASD). The oldest individual (40 years old) not diagnosed with ASD that was part of the testing group analyzed by the ASD model was also correctly identified by the ASD model as not having ASD (as shown in FIG. 10B).

Similarly, FIGS. 10C and 10D illustrate the results of a binary classification model that was developed to classify individuals with ASD vs. a control group of individuals without ASD, for the pediatric dataset. The performance of the model showcased high predictive capability to differentiate between the two groups with the performance metrics for that binary classification model as displayed in Table 5 below.

TABLE 5

| Performance Metrics | Value |
|---|---|
| AUROC (95% CI) | 0.981 (0.978-0.983) |
| Sensitivity (95% Cl) | 0.95 (0.946-0.955) |
| Specificity (95% CI) | 0.922 (0.918-0.926) |
| PPV (95% CI) | 0.881 (0.874-0.887) |
| NPV (95% CI) | 0.968 (0.965-0.971) |

FIG. 10C illustrates a histogram plot showing the distribution of age for the population with a clinical diagnosis of ASD and, also, the predicted pediatric population by the ASD model as having ASD (i.e., true positive values, that is, the population correctly predicted by the ASD model as having ASD). Further, FIG. 10D illustrates a histogram plot showing the distribution of age for the pediatric population without a clinical diagnosis of ASD and also predicted by the ASD model as not having ASD (i.e., true negative values, that is, the population correctly predicted by the ASD model as not having ASD). FIGS. 10C and 10D further indicate the strong predictive capability of the ASD model.

### Example 4

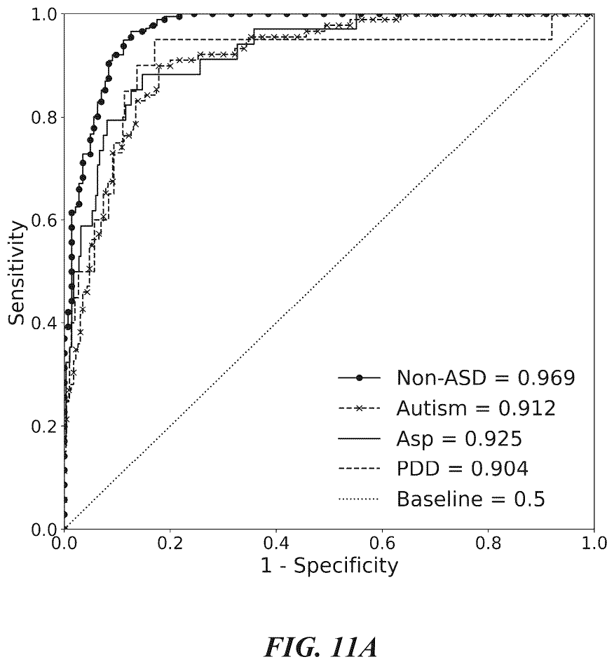

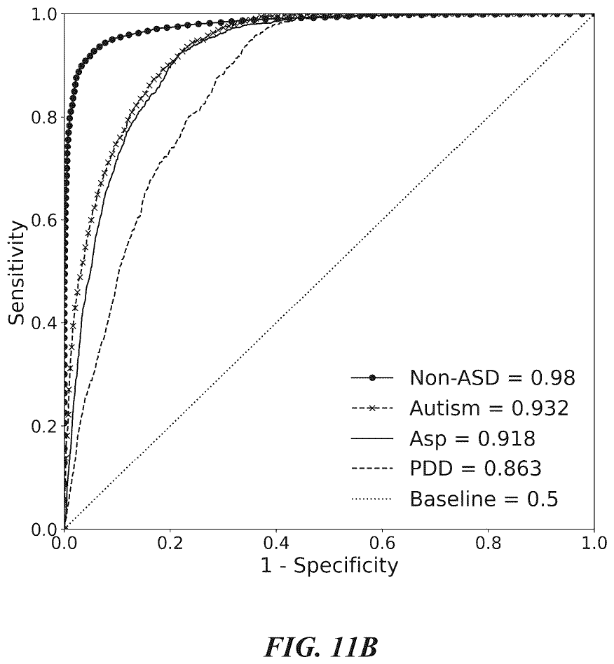

The ability of the ASD model to differentiate between subjects with no disorder, autism, Asperger syndrome, or PDD-NOS was investigated for the general population dataset and the data are displayed in FIG. 11A and Table 6. Table 6 is calculated based on the ROC curves (for the general population dataset) in FIG. 11A.

TABLE 6

| | | | Asperger | |
|---|---|---|---|---|
| | Non-ASD | Autism | Syndrome | PDD-NOS |
| AUROC | 0.969 | 0.912 | 0.925 | 0.904 |
| (95% Cl) | (0.951- | (0.877- | (0.876- | (0.798- |
| | 0.984) | 0.944) | 0.965) | 0.971) |
| Sensitivity | 0.852 | 0.854 | 0.853 | 0.85 |
| (95% CI) | (0.800- | (0.781- | (0.734- | (0.694- |
| | 0.905) | 0.927) | 0.972) | 1.006) |
| Specificity | 0.930 | 0.826 | 0.874 | 0.886 |
| (95% CI) | (0.888- | (0.777- | (0.835- | (0.85- |
| | 0.972) | 0.875) | 0.912) | 0.922) |

Referring to FIG. 11A, five (5) ROC curves are shown. The diagonal dotted line is a baseline with AUROC of 0.5. The non-ASD curve in FIG. 11A is the ROC curve that represents the ASD model's ability to differentiate a non-ASD subclass from other subclasses such as autism, Asperger syndrome and PDD-NOS, for the general population dataset. The Autism curve in FIG. 11A is the ROC curve that represents the multi-class model's ability to differentiate an Autism subclass from other subclasses such as non-ASD, Asperger syndrome and PDD-NOS, for the general population dataset. Similarly, Asp and PDD-NOS in FIG. 11A are the ROC curves that represent the ASD model's ability to differentiate the Asperger syndrome and PDD-NOS subclasses respectively from the rest of the three subclasses, for the general population dataset.

The ability of the ASD model to differentiate between subjects with no disorder, autism, Asperger syndrome, or PDD-NOS was investigated for the pediatric dataset and the data are displayed in FIG. 11B and Table 7. Table 7 is calculated based on the ROC curves (for the general population dataset) in FIG. 11B.

TABLE 7

| | | | Asperger | |
|---|---|---|---|---|
| | Non-ASD | Autism | Syndrome | PDD-NOS |
| AUROC | 0.98 | 0.932 | 0.918 | 0.863 |
| (95% Cl) | (0.978- | (0.926- | (0.911- | (0.853- |
| | 0.983) | 0.938) | 0.926) | 0.874) |
| Sensitivity | 0.858 | 0.850 | 0.851 | 0.852 |
| (95% CI) | (0.848- | (0.830- | (0.828- | (0.824- |
| | 0.868) | 0.870) | 0.874) | 0.88) |
| Specificity | 0.981 | 0.846 | 0.826 | 0.721 |
| (95% CI) | (0.976- | (0.837- | (0.817- | (0.71- |
| | 0.986) | 0.855) | 0.836) | 0.732) |

Referring to FIG. 11B, five (5) ROC curves are shown, wherein the diagonal dotted line is a baseline with AUROC of 0.5. The non-ASD curve in FIG. 11B is the ROC curve that represents the ASD model's ability to differentiate a non-ASD subclass from other subclasses such as autism, Asperger syndrome and PDD-NOS, for the pediatric dataset. The Autism curve in FIG. 11B is the ROC curve that represents the multi-class model's ability to differentiate an Autism subclass from other subclasses such as non-ASD, Asperger syndrome and PDD-NOS, for the pediatric dataset. Similarly, Asp and PDD-NOS in FIG. 11B are the ROC curves that represent the ASD model's ability to differentiate the Asperger syndrome and PDD-NOS subclasses respectively from the rest of the three subclasses, for the general population dataset.

Overall, the data in the Examples demonstrates that the ASD model can be employed with different datasets and can provide for differentiating between 2, 3, or more different sub-classes.

### Additional Embodiments

The following additional embodiments provide further examples of the subject matter disclosed herein.

A 1^{st} embodiment is a method implemented via a computing device, the method comprising receiving, by the computing device, data associated with a subject, the data associated with the subject comprising two or more of demographic data, comorbidity data, observational assessment and interview data, and medication data. The method also comprises evaluating, by the computing device, the data associated with the subject via an autism spectrum disorder (ASD) model, wherein the ASD model is configured to evaluate the data associated with the subject to determine the presence or absence of an ASD and, based upon a determination of the presence of an ASD, classify the ASD, wherein evaluation of the data associated with the subject by the ASD model yields an evaluation result, wherein the evaluation result indicates the presence or absence of the ASD.

A 2^{nd} embodiment is the method of the 1^{st} embodiment, wherein the evaluation result indicates the presence of the ASD, and wherein the evaluation result further indicates a classification of the ASD.

A 3^{rd} embodiment is the method of the 2^{nd} embodiment, wherein the classification of the ASD is one of autistic disorder, Asperger syndrome, or pervasive developmental disorder-not otherwise specified (PDD-NOS).

A 4^{th} embodiment is the method of one of the 1^{st} through the 3^{rd} embodiments, wherein the data associated with the subject comprises the demographic data, wherein the demographic data comprises age data, intelligence quotient (IQ) data, sex data, handedness data, or combinations thereof.

A 5^{th} embodiment is the method of one of the 1^{st} through the 4^{th} embodiments, wherein the data associated with the subject comprises the comorbidity data, wherein the comorbidity data comprises an indication of the presence or absence of attention deficit hyperactivity disorder (ADHD), a phobia, oppositional defiant disorder (ODD), obsessive-compulsive disorder (OCD), anxiety, generalized anxiety disorder (GAD), or combinations thereof.

A 6^{th} embodiment is the method of one of the 1^{st} through the 5^{th} embodiments, wherein the data associated with the subject comprises the observational assessment and interview data, wherein the observational assessment and interview data comprises Autism Diagnostic Instrument-Revised (ADI-R) data, Autism Diagnostic Observation Schedule (ADOS) 1st and/or 2nd Edition (ADOS and/or ADOS-2) data, Social Responsiveness Scale (SRS) data, Social Communication Questionnaire (SCQ) data, Autism Screening Questionnaire (ASQ) data, Vineland Adaptive Behavior Scale (VABS) data, Behavior Rating Inventory of Executive Function (BRIEF) data, or combinations thereof.

A 7^{th} embodiment is the method of one of the 1^{st} through the 6^{th} embodiments, wherein the data associated with the subject comprises the medication data, wherein the medication data comprises an indication of any medications used by the subject.

A 8^{th} embodiment is the method of one of the 1^{st} through the 7^{th} embodiments, wherein the data associated with the subject comprises structured data.

A 9^{th} embodiment is the method of one of the 1^{st} through the 8^{th} embodiments, wherein the ASD model is a machine-learning model selected from the group consisting of a deep learning model, a generative adversarial network model, a computational neural network model, a recurrent neural network model, a perceptron model, a classical tree machine-learning model, a decision tree type model, a regression type model, a classification model, a reinforcement learning model, and combinations thereof.

A 10^{th} embodiment is the method of the 9^{th} embodiment, wherein the machine-learning model is a gradient-boosted tree model.

An 11^{th} embodiment is the method of the 10^{th} embodiment, wherein the gradient-boosted tree model has a depth of at least 2 and not more than 7.

A 12^{th} embodiment is the method of one of the 10^{th} through the 11^{th} embodiments, wherein the gradient-boosted tree model has a depth of not more than 5.

A 13^{th} embodiment is the method of one of the 10^{th} through the 12^{th} embodiments, wherein the gradient-boosted tree model has a depth of not more than 3.

A 14^{th} embodiment is the method of one of the 10^{th} through the 13^{th} embodiments, wherein the gradient-boosted tree model comprises a plurality of decision trees.

A 15^{th} embodiment is the method of one of the 10^{th} through the 14^{th} embodiments, wherein the gradient-boosted tree model comprises at least 200 decision trees.

A 16^{th} embodiment is the method of one of the 10^{th} through the 15^{th} embodiments, wherein the gradient-boosted tree model comprises at least 300 decision trees.

A 17^{th} embodiment is the method of one of the 10^{th} through the 16^{th} embodiments, wherein the gradient-boosted tree model comprises from about 200 to about 600 decision trees.

A 18^{th} embodiment is the method of one of the 10^{th} through the 17^{th} embodiments, wherein the plurality of decision trees are weighted.

A 19^{th} embodiment is the method of one of the 10^{th} through the 18^{th} embodiments, further comprising providing therapy to the subject.

A 20^{th} embodiment is the method of the 19^{th} embodiment, wherein the therapy provided to the subject is based upon the evaluation results.

A 21^{st} embodiment is the method of the 20^{th} embodiment, wherein the therapy provided to the subject is based upon a classification of the ASD.

A 22^{nd} embodiment is the method of one of the 19^{th} through the 21^{st} embodiments, wherein the therapy is provided to the subject via the computing device, a second computing device in signal communication with the computing device, or combinations thereof.

A 23^{rd} embodiment is the method of one of the 1^{st} through the 22^{nd} embodiments, wherein the computing device comprises an edge computing device, a cloud computing device, or both.

A 24^{th} embodiment is the method of one of the 19^{th} through the 21^{st} embodiments, wherein the therapy comprises at least one of applied behavioral analysis (ABA), speech therapy, or physical therapy.

A 25^{th} embodiment is the method of one of the 1^{st} through the 24^{th} embodiments further comprising transforming the data associated with the subject into discrete numerical vectors, wherein the discrete numerical vectors are provided to the ASD model to determine the evaluation result.

A 26^{th} embodiment is the method of one of the 10^{th} through the 18^{th} embodiments further comprising (i) identifying ASD model hyperparameters, wherein the ASD model hyperparameters comprise depth and/or number of decision trees; and (ii) tuning the ASD model hyperparameters, wherein the tuning of the ASD model hyperparameters is effective to provide for an ASD model sensitivity of from about 0.75 to about 0.99.

A 27^{th} embodiment is a computing system for evaluating a subject with respect to ASD, the system comprising a computing device, the computing device comprising a processor and a non-transitory computer-readable medium, wherein the non-transitory computer-readable medium includes instructions configured to cause the processor to implement an ASD model, wherein the ASD model, when implemented via the processor, causes the computing device to receive data associated with a subject, the data associated with the subject comprising two or more of demographic data, comorbidity data, observational assessment and interview data, and medication data; evaluate the data associated with the subject via an ASD model, wherein the ASD model is configured to evaluate the data associated with the subject to determine the presence or absence of an ASD and, based upon a determination of the presence of an ASD, classify the ASD, wherein evaluation of the data associated with the subject by the ASD model yields an evaluation result, wherein the evaluation result indicates the presence or absence of the ASD.

A 28^{th} embodiment is the system of the 27^{th} embodiment, wherein the evaluation result indicates the presence of the ASD, and wherein the evaluation result further indicates a classification of the ASD.

A 29^{th} embodiment is the system of the 28^{th} embodiment, wherein the classification of the ASD is one of autistic disorder, Asperger syndrome, or pervasive developmental disorder-not otherwise specified (PDD-NOS).

A 30^{th} embodiment is the system of one of the 27^{th} through the 29^{th} embodiments, wherein the data associated with the subject comprises the demographic data, wherein the demographic data comprises age data, intelligence quotient (IQ) data, sex data, handedness data, or combinations thereof.

A 31^{st} embodiment is the system of one of the 27^{th} through the 30^{th} embodiments, wherein the data associated with the subject comprises the comorbidity data, wherein the comorbidity data comprises an indication of the presence or absence of attention deficit hyperactivity disorder (ADHD), a phobia, oppositional defiant disorder (ODD), obsessive-compulsive disorder (OCD), anxiety, generalized anxiety disorder (GAD), or combinations thereof.

A 32^{nd} embodiment is the system of one of the 27^{th} through the 31^{st} embodiments, wherein the data associated with the subject comprises the observational assessment and interview data, wherein the observational assessment and interview data comprises Autism Diagnostic Instrument-Revised (ADI-R) data, Autism Diagnostic Observation Schedule (ADOS) 1st and/or 2nd Edition (ADOS and/or ADOS-2) data, Social Responsiveness Scale (SRS) data, Social Communication Questionnaire (SCQ) data, Autism Screening Questionnaire (ASQ) data, Vineland Adaptive Behavior Scale (VABS) data, Behavior Rating Inventory of Executive Function (BRIEF) data, or combinations thereof.

A 33^{rd} embodiment is the system of one of the 27^{th} through the 32^{nd} embodiments, wherein the data associated with the subject comprises the medication data, wherein the medication data comprises an indication of any medications used by the subject.

A 34^{th} embodiment is the system of one of the 27^{th} through the 33^{rd} embodiments, wherein the data associated with the subject comprises structed data.

A 35^{th} embodiment is the system of one of the 27^{th} through the 34^{th} embodiments, wherein the ASD model is a machine-learning model selected from the group consisting of a deep learning model, a generative adversarial network model, a computational neural network model, a recurrent neural network model, a perceptron model, a classical tree machine-learning model, a decision tree type model, a regression type model, a classification model, a reinforcement learning model, and combinations thereof.

A 36^{th} embodiment is the system of the 35^{th} embodiment, wherein the machine-learning model is a gradient-boosted tree model.

A 37^{th} embodiment is the system of the 36^{th} embodiment, wherein the gradient-boosted tree model has a depth of at least 2 and not more than 7.

A 38^{th} embodiment is the system of one of the 36^{th} through the 37^{th} embodiments, wherein the gradient-boosted tree model has a depth of not more than 5.

A 39^{th} embodiment is the system of one of the 36^{th} through the 38^{th} embodiments claims A10-A12, wherein the gradient-boosted tree model has a depth of not more than 3.

A 40^{th} embodiment is the system of one of the 36^{th} through the 39^{th} embodiments, wherein the gradient-boosted tree model comprises a plurality of decision trees.

A 41^{st} embodiment is the system of one of the 36^{th} through the 40^{th} embodiments claims A10-A14, wherein the gradient-boosted tree model comprises at least 200 decision trees.

A 42^{nd} embodiment is the system of one of the 36^{th} through the 41^{st} embodiments, wherein the gradient-boosted tree model comprises at least 300 decision trees.

A 43^{rd} embodiment is the system of one of the 36^{th} through the 42^{nd} embodiments, wherein the gradient-boosted tree model comprises from about 200 to about 600 decision trees.

A 44^{th} embodiment is the system of one of the 36^{th} through the 43^{rd} embodiments, wherein the plurality of decision trees are weighted.

A 45^{th} embodiment is the system of one of the 27^{th} through the 44^{th} embodiments, wherein the ASD model, when implemented via the processor, further causes the computing device to provide therapy to the subject.

A 46^{th} embodiment is the system of the 45^{th} embodiment, wherein the therapy provided to the subject is based upon the evaluation results.

A 47^{th} embodiment is the system of the 46^{th} embodiment, wherein the therapy provided to the subject is based upon a classification of the ASD.

A 48^{th} embodiment is the system of one of the 45^{th} through the 47^{th} embodiments, wherein the therapy is provided to the subject via the computing device, a second computing device in signal communication with the computing device, or combinations thereof.

A 49^{th} embodiment is the system of one of the 27^{th} through the 48^{th} embodiments, wherein the computing device comprises an edge computing device, a cloud computing device, or both.

A 50^{th} embodiment is the system of one of the 45^{th} through the 47^{th} embodiments, wherein the therapy comprises at least one of applied behavioral analysis (ABA), speech therapy, or physical therapy.

A 51^{st} embodiment is the system of one of the 27^{th} through the 50^{th} embodiments, wherein the computing device is configured to transform the data associated with the subject into discrete numerical vectors, wherein the discrete numerical vectors are provided to the ASD model to determine the evaluation result.

A 52^{nd} embodiment is the system of one of the 36^{th} through the 44^{th} embodiments, wherein the ASD model comprises hyperparameters, wherein the hyperparameters comprise depth and/or number of decision trees; wherein the ASD model is configured to tune the hyperparameters, and wherein the tuning of the ASD model hyperparameters is effective to provide for an ASD model sensitivity of from about 0.75 to about 0.99.

A 53^{rd} embodiment is a method implemented via a computing device, the method comprising receiving, by the computing device, training data associated with a plurality of subjects, wherein at least a portion of the subjects are persons characterized as having an ASD, and wherein the training data associated with each of the subjects comprises two or more of demographic data, comorbidity data, observational assessment and interview data, and medication data. The method also comprises processing the training data associated with the plurality of subjects to yield an ASD model, wherein the ASD model is configured to evaluate data associated with a subject to determine the presence or absence of an ASD and, based upon a determination of the presence of an ASD, classify the ASD, wherein evaluation of the data associated with the subject by the ASD model yields an evaluation result, wherein the evaluation result indicates the presence or absence of the ASD.

A 54^{th} embodiment is the method of the 53^{rd} embodiment, wherein the evaluation result indicates the presence of the ASD, and wherein the evaluation result further indicates a classification of the ASD.

A 55^{th} embodiment is the method the 54^{th} embodiment, wherein the classification of the ASD is one of autistic disorder, Asperger syndrome, or pervasive developmental disorder-not otherwise specified (PDD-NOS).

A 56^{th} embodiment is the method of one of the 53^{rd} through the 55^{th} embodiments, wherein the training data associated with the plurality of subjects comprises the demographic data, wherein the demographic data comprises age data, intelligence quotient (IQ) data, sex data, handedness data, or combinations thereof.

A 57^{th} embodiment is the method of one of the 53^{rd} through the 56^{th} embodiments, wherein the training data associated with plurality of subjects comprises the comorbidity data, wherein the comorbidity data comprises an indication of the presence or absence of attention deficit hyperactivity disorder (ADHD), a phobia, oppositional defiant disorder (ODD), obsessive-compulsive disorder (OCD), anxiety, generalized anxiety disorder (GAD), or combinations thereof.

A 58^{th} embodiment is the method of one of the 53^{rd} through the 57^{th} embodiments, wherein the training data associated with the plurality of subjects comprises the observational assessment and interview data, wherein the observational assessment and interview data comprises Autism Diagnostic Instrument-Revised (ADI-R) data, Autism Diagnostic Observation Schedule (ADOS) 1st and/or 2nd Edition (ADOS and/or ADOS-2) data, Social Responsiveness Scale (SRS) data, Social Communication Questionnaire (SCQ) data, Autism Screening Questionnaire (ASQ) data, Vineland Adaptive Behavior Scale (VABS) data, Behavior Rating Inventory of Executive Function (BRIEF) data, or combinations thereof.

A 59^{th} embodiment is the method of one of the 53^{rd} through the 58^{th} embodiments, wherein the training data associated with the plurality of subjects comprises the medication data, wherein the medication data comprises an indication of any medications used by the subject.

A 60^{th} embodiment is the method of one of the 53^{rd} through the 59^{th} embodiments, wherein the training data associated with the plurality of subjects comprises structured data.

A 61^{st} embodiment is the method of one of the 53^{rd} through the 60^{th} embodiments, wherein the ASD model is a machine-learning model selected from the group consisting of a deep learning model, a generative adversarial network model, a computational neural network model, a recurrent neural network model, a perceptron model, a classical tree machine-learning model, a decision tree type model, a regression type model, a classification model, a reinforcement learning model, and combinations thereof.

A 62^{nd} embodiment is the method of the 61^{st} embodiment, wherein the machine-learning model is a gradient-boosted tree model.

A 63^{rd} embodiment is the method of the 62^{nd} embodiment, wherein the gradient-boosted tree model has a depth of at least 2 and not more than 7.

A 64^{th} embodiment is the method of one of the 62^{nd} through the 63^{rd} embodiments, wherein the gradient-boosted tree model has a depth of not more than 5.

A 65^{th} embodiment is the method of one of the 62^{nd} through the 64^{th} embodiments, wherein the gradient-boosted tree model has a depth of not more than 3.

A 66^{th} embodiment is the method of one of the 62^{nd} through the 65^{th} embodiments, wherein the gradient-boosted tree model comprises a plurality of decision trees.

A 67^{th} embodiment is the method of one of the 62^{nd} through the 66^{th} embodiments, wherein the gradient-boosted tree model comprises at least 200 decision trees.

A 68^{th} embodiment is the method of one of the 62^{nd} through the 67^{th} embodiments, wherein the gradient-boosted tree model comprises at least 300 decision trees.

A 69^{th} embodiment is the method of one of the 62^{nd} through the 68^{th} embodiments, wherein the gradient-boosted tree model comprises from about 200 to about 600 decision trees.

A 70^{th} embodiment is the method of one of the 62^{nd} through the 69^{th} embodiments, wherein the plurality of decision trees are weighted.

A 71^{st} embodiment is the method of one of the 53^{rd} through the 70^{th} embodiments, wherein the computing device comprises an edge computing device, a cloud computing device, or both.

A 72^{nd} embodiment is the method of one of the 53^{rd} through the 71^{st} embodiments, further comprising transforming the training data associated with the plurality of subjects into discrete numerical vectors, wherein the discrete numerical vectors are processed, by the computing device, to yield the ASD model.

A 73^{rd} embodiment is the method of one of the 53^{rd} through the 72^{nd} embodiments, wherein the ASD model is further configured to process data associated with the subject that has been transformed to yield discrete numerical vectors, wherein the ASD model is configured to process the discrete numerical vectors to determine the evaluation result.

A 74^{th} embodiment is the method of one of the 62^{nd} through the 70^{th} embodiments, further comprising (i) identifying ASD model hyperparameters, wherein the ASD model hyperparameters comprise depth and/or number of decision trees; and (ii) tuning the ASD model hyperparameters, wherein the tuning of the ASD model hyperparameters is effective to provide for an ASD model sensitivity of from about 0.75 to about 0.99.

A 75^{th} embodiment is a method implemented via a computing device, the method comprising: receiving, by the computing device, data associated with a subject, the data associated with the subject comprising two or more of demographic data, comorbidity data, observational assessment and interview data, and medication data; and evaluating, by the computing device, the data associated with the subject via an ASD model, wherein the ASD model is configured to evaluate the data associated with the subject to yield an evaluation result, and wherein the evaluation result indicates a finding of non-ASD, a finding of autistic disorder, a finding of Asperger syndrome, or a finding of pervasive developmental disorder-not otherwise specified (PDD-NOS) for the subject.

A 76^{th} embodiment is the method of the 75^{th} embodiment, wherein the data associated with the subject comprises the demographic data, wherein the demographic data comprises age data, intelligence quotient (IQ) data, sex data, handedness data, or combinations thereof.

A 77^{th} embodiment is the method of one of the 75^{th} through the 76^{th} embodiments, wherein the data associated with the subject comprises the comorbidity data, wherein the comorbidity data comprises an indication of the presence or absence of attention deficit hyperactivity disorder (ADHD), a phobia, oppositional defiant disorder (ODD), obsessive-compulsive disorder (OCD), anxiety, generalized anxiety disorder (GAD), or combinations thereof.

A 78^{th} embodiment is the method of one of the 75^{th} through the 77^{th} embodiments, wherein the data associated with the subject comprises the observational assessment and interview data, wherein the observational assessment and interview data comprises Autism Diagnostic Instrument-Revised (ADI-R) data, Autism Diagnostic Observation Schedule (ADOS) 1st and/or 2nd Edition (ADOS and/or ADOS-2) data, Social Responsiveness Scale (SRS) data, Social Communication Questionnaire (SCQ) data, Autism Screening Questionnaire (ASQ) data, Vineland Adaptive Behavior Scale (VABS) data, Behavior Rating Inventory of Executive Function (BRIEF) data, or combinations thereof.

A 79^{th} embodiment is the method of one of the 75^{th} through the 78^{th} embodiments, wherein the data associated with the subject comprises the medication data, wherein the medication data comprises an indication of any medications used by the subject.

An 80^{th} embodiment is the method of one of the 75^{th} through the 79^{th} embodiments, wherein the data associated with the subject comprises structured data.

An 81^{st} embodiment is the method of one of the 75^{th} through the 80^{th} embodiments, wherein the ASD model is a machine-learning model selected from the group consisting of a deep learning model, a generative adversarial network model, a computational neural network model, a recurrent neural network model, a perceptron model, a classical tree machine-learning model, a decision tree type model, a regression type model, a classification model, a reinforcement learning model, and combinations thereof.

An 82^{nd} embodiment is the method of the 81^{st} embodiment, wherein the machine-learning model is a gradient-boosted tree model.

An 83^{rd} embodiment is the method of the 82^{nd} embodiment, wherein the gradient-boosted tree model has a depth of at least 2 and not more than 7.

An 84^{th} embodiment is the method of one of the 82^{nd} through the 83^{rd} embodiments, wherein the gradient-boosted tree model has a depth of not more than 5.

An 85^{th} embodiment is the C11. The method of one of the 82^{nd} through the 84^{th} embodiments, wherein the gradient-boosted tree model has a depth of not more than 3.

An 86^{th} embodiment is the method of one of the 82^{nd} through the 85^{th} embodiments, wherein the gradient-boosted tree model comprises a plurality of decision trees.

An 87^{th} embodiment is the method of one of the 82^{nd} through the 86^{th} embodiments, wherein the gradient-boosted tree model comprises at least 200 decision trees.

An 88^{th} embodiment is the method of one of the 82^{nd} through the 87^{th} embodiments, wherein the gradient-boosted tree model comprises at least 300 decision trees.

An 89^{th} embodiment is the C15. The method of one of the 82^{nd} through the 88^{th} embodiments, wherein the gradient-boosted tree model comprises from about 200 to about 600 decision trees.

A 90^{th} embodiment is the method of one of the 82^{nd} through the 89^{th} embodiments, wherein the plurality of decision trees are weighted.

A 91^{st} embodiment is the method of one of the 75^{th} through the 90^{th} embodiments, further comprising providing therapy to the subject.

A 92^{nd} embodiment is the method of the 91^{st} embodiment, wherein the therapy provided to the subject is based upon the evaluation results.

A 93^{rd} embodiment is the method of the 91^{st} embodiment, wherein the therapy provided to the subject is based the finding of autistic disorder, the finding of Asperger syndrome, or the finding of PDD-NOS.

A 94^{th} embodiment is the method of one of the 91^{st} through the 93^{rd} embodiments, wherein the therapy is provided to the subject via the computing device, a second computing device in signal communication with the computing device, or combinations thereof.

A 95^{th} embodiment is the method of one of the 75^{th} through the 94^{th} embodiments, wherein the computing device comprises an edge computing device, a cloud computing device, or both.

A 96^{th} embodiment is the method of one of the 91^{st} through the 93^{rd} embodiments, wherein the therapy comprises at least one of applied behavioral analysis (ABA), speech therapy, or physical therapy.

A 97^{th} embodiment is the method of one of the 75^{th} through the 96^{th} embodiments further comprising transforming the data associated with the subject into discrete numerical vectors, wherein the discrete numerical vectors are provided to the ASD model to determine the evaluation result.

A 98^{th} embodiment is the method of one of the 82^{nd} through the 90^{th} embodiments further comprising (i) identifying ASD model hyperparameters, wherein the ASD model hyperparameters comprise depth and/or number of decision trees; and (ii) tuning the ASD model hyperparameters, wherein the tuning of the ASD model hyperparameters is effective to provide for an ASD model sensitivity of from about 0.75 to about 0.99.

While embodiments of the disclosure have been shown and described, modifications thereof can be made without departing from the spirit and teachings of the invention. The embodiments and examples described herein are exemplary only, and are not intended to be limiting. Many variations and modifications of the invention disclosed herein are possible and are within the scope of the invention.

Accordingly, the scope of protection is not limited by the description set out above but is only limited by the claims which follow, that scope including all equivalents of the subject matter of the claims. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). For example, whenever a numerical range with a lower limit, Rₗ, and an upper limit, Rᵤ, is disclosed, any number falling within the range is specifically disclosed. In particular, the following numbers within the range are specifically disclosed: R=Rₗ+k*(Rᵤ−Rₗ), wherein k is a variable ranging from 1 percent to 100 percent with a 1 percent increment, i.e., k is 1 percent, 2 percent, 3 percent, 4 percent, 5 percent, . . . , 50 percent, 51 percent, 52 percent, . . . , 95 percent, 96 percent, 97 percent, 98 percent, 99 percent, or 100 percent. Moreover, any numerical range defined by two R numbers as defined in the above is also specifically disclosed. Also, use of the term “about” with respect to a disclosed value or other quantification should be understood to include those values proximate to the disclosed value, for example, deviating from the disclosed value by ±0.1% of the disclosed value, or ±0.5%, or ±1%, or ±2%, or ±3%, or ±4%, or ±5%, or ±6%, or ±7%, or ±8%, or ±9%, or ±10%, as contextually appropriate. Each and every claim is incorporated into the specification as an embodiment of the present invention. Thus, the claims are a further description and are in addition to the detailed description of the present invention. The disclosures of all patents, patent applications, and publications cited herein are hereby incorporated by reference.

What is claimed is:

1. A method implemented via a computing device, the method comprising:
receiving, by the computing device, data associated with a subject, the data associated with the subject comprising two or more of demographic data, comorbidity data, observational assessment and interview data, and medication data; and
evaluating, by the computing device, the data associated with the subject via an autism spectrum disorder (ASD) model, wherein the ASD model is configured to evaluate the data associated with the subject to determine the presence or absence of an ASD and, based upon a determination of the presence of an ASD, classify the ASD, wherein evaluation of the data associated with the subject by the ASD model yields an evaluation result, wherein the evaluation result indicates the presence or absence of the ASD.

2. The method of claim 1, wherein the evaluation result indicates the presence of the ASD, and wherein the evaluation result further indicates a classification of the ASD.

3. The method of claim 2, wherein the classification of the ASD is one of autistic disorder, Asperger syndrome, or pervasive developmental disorder-not otherwise specified (PDD-NOS).

4. The method of claim 1, wherein the data associated with the subject comprises the demographic data, wherein the demographic data comprises age data, intelligence quotient (IQ) data, sex data, handedness data, or combinations thereof.

5. The method of claim 1, wherein the data associated with the subject comprises the comorbidity data, wherein the comorbidity data comprises an indication of the presence or absence of attention deficit hyperactivity disorder (ADHD), a phobia, oppositional defiant disorder (ODD), obsessive-compulsive disorder (OCD), anxiety, generalized anxiety disorder (GAD), or combinations thereof.

6. The method of claim 1, wherein the data associated with the subject comprises the observational assessment and interview data, wherein the observational assessment and interview data comprises Autism Diagnostic Instrument-Revised (ADI-R) data, Autism Diagnostic Observation Schedule (ADOS) 1^{st} and/or 2^{nd} Edition (ADOS and/or ADOS-2) data, Social Responsiveness Scale (SRS) data, Social Communication Questionnaire (SCQ) data, Autism Screening Questionnaire (ASQ) data, Vineland Adaptive Behavior Scale (VABS) data, Behavior Rating Inventory of Executive Function (BRIEF) data, or combinations thereof.

7. The method of claim 1, wherein the data associated with the subject comprises the medication data, wherein the medication data comprises an indication of any medications used by the subject.

8. The method of claim 1, wherein the ASD model is a machine-learning model selected from the group consisting of a deep learning model, a generative adversarial network model, a computational neural network model, a recurrent neural network model, a perceptron model, a classical tree machine-learning model, a decision tree type model, a regression type model, a classification model, a reinforcement learning model, and combinations thereof.

9. The method of claim 8, wherein the machine-learning model is a gradient-boosted tree model having a depth of at least 2 and not more than 7.

10. The method of claim 9, wherein the gradient-boosted tree model has a depth of not more than 3.

11. The method of claim 9, wherein the gradient-boosted tree model comprises at least 200 decision trees.

12. The method of claim 9, wherein the gradient-boosted tree model comprises from about 200 to about 600 decision trees.

13. The method of claim 9, wherein the plurality of decision trees are weighted.

14. A computing system for evaluating a subject with respect to autism spectrum disorder (ASD), the system comprising:
a computing device, the computing device comprising a processor and a non-transitory computer-readable medium, wherein the non-transitory computer-readable medium includes instructions configured to cause the processor to implement an ASD model, wherein the ASD model, when implemented via the processor, causes the computing device to:
receive data associated with a subject, the data associated with the subject comprising two or more of demographic data, comorbidity data, observational assessment and interview data, and medication data;
evaluate the data associated with the subject via an ASD model, wherein the ASD model is configured to evaluate the data associated with the subject to determine the presence or absence of an ASD and, based upon a determination of the presence of an ASD, classify the ASD, wherein evaluation of the data associated with the subject by the ASD model yields an evaluation result, wherein the evaluation result indicates the presence or absence of the ASD.

15. The system of claim 14, wherein the evaluation result indicates the presence of the ASD, and wherein the evaluation result further indicates a classification of the ASD, wherein the classification of the ASD is one of autistic disorder, Asperger syndrome, or pervasive developmental disorder-not otherwise specified (PDD-NOS).

16. The system of claim 14, wherein the ASD model is a machine-learning model selected from the group consisting of a deep learning model, a generative adversarial network model, a computational neural network model, a recurrent neural network model, a perceptron model, a classical tree machine-learning model, a decision tree type model, a regression type model, a classification model, a reinforcement learning model, and combinations thereof.

17. The system of claim 16, wherein the machine-learning model is a gradient-boosted tree model having a depth of at least 2 and not more than 7, and wherein the gradient-boosted tree model comprises at least 200 weighted decision trees.

18. A method implemented via a computing device, the method comprising:
receiving, by the computing device, data associated with a subject, the data associated with the subject comprising two or more of demographic data, comorbidity data, observational assessment and interview data, and medication data; and
evaluating, by the computing device, the data associated with the subject via an autism spectrum disorder (ASD) model, wherein the ASD model is configured to evaluate the data associated with the subject to yield an evaluation result, and wherein the evaluation result indicates a finding of non-ASD, a finding of autistic disorder, a finding of Asperger syndrome, or a finding of pervasive developmental disorder-not otherwise specified (PDD-NOS) for the subject.

19. The method of claim 18, wherein the ASD model is a machine-learning model selected from the group consisting of a deep learning model, a generative adversarial network model, a computational neural network model, a recurrent neural network model, a perceptron model, a classical tree machine-learning model, a decision tree type model, a regression type model, a classification model, a reinforcement learning model, and combinations thereof.

20. The method of claim 19, wherein the machine-learning model is a gradient-boosted tree model having a depth of at least 2 and not more than 7, and wherein the gradient-boosted tree model comprises from about 200 to about 600 weighted decision trees.
